(19) United States
(12) Patent Application Publication
(10) Pub. No.: US 2014/0249424 A1
(43) Pub. Date: Sep. 4, 2014

(54) CARDIOVASCULAR PULSE WAVE ANALYSIS METHOD AND SYSTEM

(71) Applicant: University of Winnipeg, Winnipeg (CA)

(72) Inventors: Zhaopeng Fan, Winnipeg (CA); Michael Gong Zhang, Winnipeg (CA); Simon Xin Meng Liao, Winnipeg (CA)

(73) Assignee: University of Winnipeg, Winnipeg (CA)

(21) Appl. No.: 14/096,872

(22) Filed: Dec. 4, 2013

Related U.S. Application Data
(60) Provisional application No. 61/733,298, filed on Dec. 4, 2012.

(30) Foreign Application Priority Data
Dec. 4, 2012 (CA) 2798337

(51) Int. Cl. A61B 5/00; A61B 5/024

(52) U.S. Cl. CPC A61B 5/7282; A61B 5/02433; A61B 5/6826; A61B 5/726; A61B 5/7264; A61B 5/742
USPC 600/473; 600/501

(57) ABSTRACT

Factor retrieving is a major approach for pulse wave analysis. Stiffness index and cardiac output are widely used factors for cardiac risk detection. Research has been done on clinical pulse wave data which are collected by pulse oximeter. The result shows that collected factors have a positive correlation with certain cardiac risks. Some adjustments have been applied on the algorithms that increase the significance. In addition to the factor based analysis, other signal processing techniques for pulse waveforms are included such as bispectrum estimation, Wavelet transform, and weighted dynamic time warping. Bispectrum estimation and Wavelet transform have meaningful features of pulse waveforms with some special shapes. Weighted dynamic time warping compares the similarity of waveforms. It also includes medical significance into the calculation by adjusting the weight vector. This algorithm has higher accuracy when providing more samples to compare. The factor based analysis and waveform analysis compose an analytic model which can be used for risk evaluation, classification and disease detection.

### CROSS REFERENCE TO RELATED APPLICATION

This application claims benefit to U.S. provisional patent application Ser. No. 61/733,298, filed Dec. 4, 2012, the contents of which are incorporated herein in their entirety.

### FIELD OF INVENTION

The present invention relates to cardiovascular testing and analysis, and more specifically, to a method of and system for performing cardiovascular risk assessment and disease detection using pulse wave analysis.

### BACKGROUND OF THE INVENTION

Cardiovascular diseases are one of the leading causes of death in the world. An estimated 17.3 million deaths, which is 30% of global deaths, were caused by cardiovascular diseases in 2008 according to the World Health Organization. It is therefore clear that research into cardiovascular diseases can save lives and benefit public health.

A number of approaches to cardiovascular testing and analysis have been proposed, but none of have been found to be straightforward and effective.

For example, Doppler Vascular Ultrasound uses the following approach:
two-dimensional ultrasound and m-scan echocardiogram;
calculating the sectional area A(cm²) of the aorta based on the diameter;
monitoring the speed wave of blood flow at the position above sternum; and
calculating the cardiac output Q=A×V×t based derivative of the speed wave.
   While this technical provides good visual information, it has several disadvantages such as low resolution images, requiring a special posture to monitor, and needing a health care professional to operate.

Radionuclide cardiac angiography uses the following:
inputting an indicator by intravenous infusion;
locating the sensor at precordium-precordia;
calculating the radiocardiogram E and area A by monitoring indicators; and
based on the isotope dosimetry C, calculating the cardiac output as Q=E×C/A×V.
   This approach has limitations in the device and indicator that are required, and that a professional operator is needed.

An analysis of electrocardiograms (ECG), carotid displacement curve (CAR), phonocardiogram (PCG), and apexcardiogram (ACG) may also be done. Useful information may be obtained by analyzing these signals but they must be synchronized to achieve accurate results. This approach also requires sophisticated equipment with many leads and sensors, as well as assistance from health care professions.

A rheocardiogram (impedance cardiogram) may also be used to record the changes in body's electrical conductivity, but this is a complex operation that is not reliable, sometimes with over 25% variance.

Thus, there is for an improved method of and system for performing cardiovascular risk assessment and disease detection.

### SUMMARY OF THE INVENTION

It is an object of the invention to provide an improved method of and system for performing cardiovascular risk assessment and disease detection.

One embodiment of the invention comprises a system which will:

1. obtain a person's pulse signals from one of his/her fingers by using a pulse wave receiver;
2. apply factor-based analysis and/or waveform-based analysis of the pulse wave data to those pulse signals; and
3. report the analyzed results, such as cardiac risks, in real time.
The system is easy to use, and the computing time to provide the pulse analysis results is negligible. With a wireless pulse receiver, the system can be executed on desktop and laptop computers, iPads, Smart phones, and similar computing devices.

Another embodiment of the invention comprises a method of cardiovascular analysis comprising collecting and storing cardiovascular pulse wave data over time, and performing factor-based analysis and/or waveform-based analysis of said stored cardiovascular pulse wave data. These analysis techniques comprise in particular executing a stiffness index algorithm adjusted for pulse rate, and executing a weighted dynamic time warping algorithm. An exemplary algorithm for implementing such a method of the invention is presented in the flow chart of FIG. 6.1.

Weighted DTW (dynamic time warping) algorithms have been used in the past, but the weighted DTW algorithm described herein is an advancement over prior methods, which results in a very beneficial and unexpected improvement. Another major contribution of the invention comprises a combined model for cardiovascular disease evaluation via pulse wave analysis. Per Table 5.1 and FIG. 6.1, a combined pulse wave analysis model for cardiovascular disease detection is described, which comprises extraction and reorganization by different algorithms. The combined method similarly results in a very beneficial and unexpected improvement over the prior art.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

These and other features of the invention will become more apparent from the following description in which reference is made to the appended drawings wherein:

1.1 Stiffness index calculated by special points
1.2 Variation for continue waveforms
1.3 Waveform classification based on notch
1.4 Bates' classification for pulse waveform
2.1 Pulse wave signal at wrist
2.2 Infra-red sensor
2.3 Pulse wave signal at finger
2.4 AD converter
2.5 High-pass filtering
2.6 Age groups for all subjects
3.1 Stiffness index is related to the time delay between the systolic and diastolic components of the waveform and the subjects height [54]
3.2 Cardiac output calculation from pulse wave [44]
3.3 One-dimensional Continuous Wavelet Transform of pulse waveforms
3.4 One dimensional continuous Morlet wavelet transform of pulse waveforms
3.5 Distance of two waveforms
4.1 Stiffness index by age
4.2 Stiffness index by Systolic Blood Pressure
4.3 Stiffness index for no risk group and patient group
4.4 Adjusted stiffness index has higher correlation with Age
4.5 Histograms of pulse waveforms
4.6 Bispectrum estimation for a normal waveform
4.7 Bispectrum estimation for typical waveforms with old myocardial infarction
4.8 Bispectrum estimation for Arrhythmia pulse waveform
4.9 Wavelet transform for normal waveform
4.10 Wavelet transform for waveform with abnormal ending
4.11 Wavelet transform of high risk pulse waveform
4.12 Matrix of point to point distance between two pulse waveforms
4.13 Warping waveforms based on the dynamic time warping
4.14 Waveform of 28 years old male
4.15 Waveform of 37 years old male
4.16 Waveform of 58 years old female without cardiovascular diseases record in medical history
4.17 Sample waveforms used for classification
5.1 Pulse wave from a patient with acute anterior myocardial infarction
5.2 Waveform analysis for patient with acute anterior myocardial infarction
5.3 Waveforms detected with less distance to the sample wave
5.4 Pulse wave for patient with Old myocardial infarction and degenerative valvular disease
5.5 Waveform analysis for patient with old myocardial infarction
5.6 Samples of wave in category of old myocardial infarction
5.7 Pulse wave for a patient with Ventricular aneurysm
5.8 Waveform analysis for patient with ventricular aneurysm
5.9 Samples for pulse waves in the category with ventricular aneurysm
5.10 Pulse wave for Dilated cardiomyopathy
5.11 Waveform analysis for patient with dilated cardiomyopathy
5.12 Samples for pulse waves in the category of dilated cardiomyopathy
5.13 Pulse wave from a patient with coronary artery spasm
5.14 Waveform analysis for patient with coronary artery spasm
5.15 Pulse waveform for patient with diastolic hypertension
5.16 Waveform analysis for patient with coronary artery spasm
5.17 Pulse waveform for patient with heart failure
5.18 Waveform analysis for patient with heart failure
5.19 Pulse wave form patient with premature
5.20 Waveform analysis for patient with premature
5.21 Pulse wave form patient with Sinoatrial block
5.22 Waveform analysis for patient with Sinoatrial block

FIG. 6.1 presents a flow chart of an exemplary Pulse Wave Analysis Model

FIG. 6.2 presents an exemplary pulse wave, identifying wave portions which may be associated with different weightings under a weighted dynamic time warping analysis.

### DETAILED DESCRIPTION

As explained above, recent attempts at performing cardiovascular testing and analysis have proven to be inadequate. Preferred systems and methods which address one or more of the problems known in the art are described hereinafter, by way of particular examples. It will be apparent to persons skilled in the art that a number of variations and modifications can be made without departing from the scope of the invention as defined in the claims.

### Chapter 1

### Introduction

### 1.1 Background

Cardiovascular diseases are the top reason for death in the world [86]. An estimated 17.3 million deaths, which is 30% of global death, were caused by cardiovascular diseases in 2008 according to World Health Organization [84]. Research of cardiovascular diseases can save lives and benefit public health.

The terminology cardiovascular refers to the heart and blood vessels that provide nutrition and oxygen to tissues of body and remove metabolites from them [83]. Cardiovascular system has two major functional parts: central circulation system and peripheral circulation system. Central circulation includes the pulmonary circulation and the heart from where the pulse wave is generated. Peripheral circulation is the path that the blood goes from and to the heart [35]. Pulse wave can be detected by measuring pressure of the arteries, which include elastic arteries, medium muscular arteries, small arteries, and arterioles. The typical muscular artery has three layers: tunica intima (inner layer), tunica media (middle layer), and tunica adventitia (outer layer) [40]. The physical properties of arteries are highly nonlinear [43]. It depends on the contents of arterial wall: where collagen, elastin and protein are located in the arteries. Functional and structural changes in the arterial wall can be used as an early marker for the hypertensive and cardiovascular diseases [17].

Blood flow is the key to monitor the cardiovascular health since it is generated and restricted within the cardiovascular system [11]. Currently the most widely used method for haemodynamic parameters detecting is invasive thermodilution method [31] [68]. Impedance-cardiography is the most commonly used non-invasive method [70]. However, there are also some shortcomings for Impedance-cardiography. Multiple sensors are used in the test and each sensor has two electrodes. A wide area from neck to breast needs to be exposed so that the sensors can be placed onto predefined areas [70]. It is too complex for a clinical routine check using Impedance-cardiography. Pulse wave analysis is a quick, convenient, and innovative method for cardiovascular health screening that is suitable for both the clinical environment and home monitoring situations [87].

Pulse wave is a critical signal of cardiovascular health. It comes directly from heart to the vascular system. As a pulse is transmitted, reactions occur at different blood vessels levels. Other conditions such as resistance of blood vessel, elasticity of vessel wall, and blood viscosity have direct impacts on pulse wave [51]. Pathological changes affect the pulse wave in different ways: strength, reaction and frequency. Pulse wave provides abundant and reliable information about the cardiovascular system.

Pulse wave can be recorded by a set of time series data and represented as a diagram which is called pulse waveform or pulse wave for short.

Gathering pulse wave at wrist has been a major diagnosis method in China since about 500 BC. Emperor Huangdi is said to have summarized techniques about pulse diagnosis. Physicians used palpation of the pulse as a diagnostic tool during examinations. Greeks started to notice the rhythm, strength and velocity at 400BC [4]. In 300AD, Mai Jing, a Chinese book about pulse diagnosis, categorized pulse into 24 types and became the first systematic literature about the pulse [78]. Struthius described a method to watch the pulse wave by putting a leaf on the artery, which is considered early pulse wave monitoring. In 1860, Etienne Jules Mary invented a level based sphygmograph to measure the pulse rate. It was the first device that could actually record the pulse wave. Frederick Akbar Mohamed observed normal radial pressure wave and carotid pulse wave to find the normal waveform. He also stated the differences between those waveforms and concluded the special effect on the radial waveform that was caused by high blood pressure [48] [50]. The result helps to learn the natural history of essential hypertension. The effects of arterial degeneration by aging on the pulse wave were also shown in his work [49]. This theory has been used in the life insurance field since nineteenth century [64].

Pulse wave analysis was based on basic mathematic algorithms at that time: dividing the waveform into ascending phase and descending phase, calculating the height or area of the wave [59]. Recently, calculus, hemodynamic, biomathematics, and pattern recognition techniques have been used in pulse wave analysis [51]. However, utilizing the classic pulse theory with current techniques is still a challenging task.

### 1.2 Pulse Wave Analysis

Arterial pulse is considered the most fundamental life signal in medicine, which has been used since ancient time [4] [59]. With the help of new information technology, pulse wave analysis has been used to detect many risks of cardiovascular health especially cases involving arterial stiffness [59].

Pulse wave is non-invasive, easy and safe to get. But using pulse wave data directly for cardiovascular system evaluation is unreliable since there are always changing haemodynamic conditions. So, there are lots of research in the area of pulse wave analysis with signal processing techniques [42]. When considering related conditions, pulse wave analysis can achieve high accuracy.

Most recent research indicates positive results to pulse wave analysis when comparing with standard methods. Pathophysiological Laboratory Netherlands did a study on continuous cardiac output monitoring with pulse wave during cardiac surgery [39]. Cardiac output was measured 8 to 12 times during the operation with pulse wave and thermodilution. The result showed linear correlation between the two methods. The cardiac output calculated by pulse wave is accurate even when heart rate, blood pressure and total peripheral resistance changed.

To reduce the effects of other factors, pulse wave analysis was tested among different research groups. In 1999, Rodig G. picked two groups of patients based on ejection fraction: 13 patients in testing group with ejection fraction greater than 45% and 13 patients in control group with ejection fraction less than 45%. Both pulse wave and thermodilution technique had been used to calculate the cardiac output 12 times during the surgery. The mean differences for cardiac output did not differ in either group [66]. The differences became significant when systemic vascular resistance increased by 60% at the early period after operation. The paper suggested that pulse wave analysis is a comparable method to thermodilution techniques during the surgery. Calibration of the device will help to achieve a more accurate result.

Berton suggested that patients with weak pulse waveform or arrhythmia should avoid using the result of pulse wave since it is unreliable in such conditions [7].

Early detection of cardiovascular diseases is one of the most important uses for pulse wave monitoring [17]. The convenient noninvasive technique makes it extremely suitable for being applied at community levels. Factors derived from pulse wave analysis have been used to detect hypertension and coronary artery diseases. For example, reduced compliance of arteries may result in losing the diastolic component of pulse wave. Studies show that pulse wave may be an early marker for those diseases and a guide for health care professional personnels during the therapy [17].

There were two major streams for pulse wave analysis: point based analysis and area based analysis [59]. Point based analysis is usually designed for a specific risk factor. It picks up top and bottom points from different components of the waveform or derivative curve [54]. Then the calculation is conducted pertaining to the medical significance of those points. Stiffness index and pulse wave velocity are well-known factors in this category [54].

Artery stiffness is related to age and atherosclerosis [9]. Two of the leading reasons for death in the developed countries nowadays, myocardial infarction and stroke, are direct consequence of atherosclerosis [73]. Arterial stiffness is an indicator of increased risk of cardiovascular disease. Among many new methods to detect arterial stiffness, pulse wave analysis is a method with a promising result [54].

Total arterial compliance and increased central Pulse Wave Velocity are associated with arterial wall stiffening. They are recognized as the dominant risk factors for cardiovascular disease [8]. Pulse wave velocity is the velocity of the pulse pressure. The blood goes at a speed of more than one meter per second in the aorta and slow down to several mm per second at peripheral network. The pulse wave velocity is much faster than that. Normal pulse wave velocity has the range from 5 meters per second to 15 meters per second [58].

Since pulse pressure and pulse wave velocity are closely linked to cardiovascular morbidity, some non-invasive methods to assess arterial stiffness based on pulse wave velocity have been introduced [74]. However, these methods need to measure the difference of the centre artery pulse and the reflected pulse wave, which is a complicated process. On the other hand, the digital volume pulse can be obtained simply by measuring the artery pulse, which becomes attractive to analyze [53].

Millasseau Sandrine C. demonstrated that arterial stiffness, as measured by peripheral pulse wave analysis, is correlated with the measurement of central aortic stiffness and pulse wave velocity between carotid and femoral artery in 2002. This is considered a reliable method in assessment of cardiovascular pathologic changes for adults. He introduced the stiffness index (SI), which was derived from the pulse wave analysis for arterial stiffness assessment and was correlated with pulse wave velocity (r=0.65, P<0.0001). This is an effective non-invasive method for evaluating arterial stiffness [54].

Pulse wave velocity is the gold standard for arterial stiffness diagnosis [1]. Research shows that the stiffness index has equivalent output to pulse wave velocity [54]. Stiffness index uses the refection of the pulse as the second source to get the time difference without the use of additional sensors, which makes it more applicable to Home Monitoring System. As shown in FIG. 1.1, the systolic top shows the time that pulse reaches the finger; diastolic top represents the time that pulse reflection reaches the finger [54]. The distance that pulse goes through has a direct relationship with the height of the subject. SI can be calculated by

$$S I = \frac{h}{Δ {T}_{DVP}} \qquad ( 1.1 )$$

where h is subject's height and ΔT_{DVP} is time interval between systolic component and diastolic component.

Area Based analysis specializes in blood volume monitoring such as cardiac output. The attempt for getting cardiac output from pulse wave started more than one hundred years ago [23]. The pulse waveform is the result of interaction between stroke volume and artery resistance. Building the model of the arterial tree helped the calculation of cardiac output from pulse wave. The simplest model used in clinic studies contains a single resistance. Other elements should be involved in the calculation including a capacitance element and a resistance element [16].

Not all of the models have reliable results. Even some widely used ones can only work in specific environments. Windkessel Model consists of four elements: left ventricle, aortic valve, arterial vascular compartment and peripheral flow pathway [75]. Test of the model in normotensive and hypertensive subjects shows that the model is only valid when the pressure wave speed is high enough without reaction sites.

Cardiac Index is an important parameter representing the ratio of the cardiac output over body surface area [83]. Thomas W. Felbinger compared the cardiac index value among pulmonary artery thermodilution, arterial thermodilution, and pulse wave analysis for critically ill patients in the year 2005 [27]. The mean difference among three methods is −1.01% and standard derivation is 6.51%. The pulse wave provides clinically acceptable accuracy according to Thomas' research.

In addition to long term monitoring, pulse wave analysis is also useful for emergency environment since cardiac function can be evaluated within several seconds.

Many factors should be included in the evaluation of pulse wave data because pulse wave is the result of different systems working together. Pulse rate is equal to heart rate at most situations [33]. Cardiac output is a major factor for cardiac function [66]. It is used to calculate some other important factors such as stroke volume, stroke index, and cardiac index. Pulse pressure, stiffness index are representation of blood vessel condition. The lung function and microcirculation condition also have impact on the pulse wave [88] [55].

The calculations based on the special points or area are very sensitive for risk detection. They use simple algorithms to achieve the balance of performance and accuracy. But it is not enough to evaluate the overall cardiovascular condition by one or two pulse wave factors [82]. Analyzing pulse waveform instead of retrieving factors from pulse wave is considerable way to evaluate overall cardiovascular health.

Pulse wave data are treated as time series data in the computer system: continuous data values with constant sampling rate. Bispectrum can give estimation with statictical analysis in frequency domain [56]. Wavelet analysis uses Wavelet as basic component. The calculation includes Fourier transform and time domain analysis [76]. Dynamic time warping is an algorithm to compare the similarity of two time serial data, with some very successful application in speech recognition [6]. These three algorithms are used in proposed pulse wave analysis model.

### 1.3 Waveform Classification

According to clinical data in this thesis, pulse wave is relatively stable under the testing conditions: subject sitting in a quiet environment and keeping calm. The pulse wave analysis result is highly consistent in this condition. The similarity of pulse waveforms does not change significantly under similar cardiovascular health condition even the heart rate and pulse strength changes, so waveform analysis can be applied in different scenarios. FIG. 1.2 from O'Rourke, M F's research in 2001 also shows that pulse waveforms have low variation in same pulse wave data [59].

There are several classification systems for the pulse wave. In 1973, Tomas R. Dawber treated the notch as the indicator and classifies pulse wave into four categories as FIG. 1.3 [21]:
Class I: a distinct incisura is inscribed on the downward slope of the pulse wave
Class II: No incisura develops but the line of descent becomes horizontal
Class III: No notch is present but a well-defined change in the angle of descent is observed
Class IV: No evidence of a notch is seen

The classification focuses on the notch of the waveform which is considered as the indicator of arterial stiffness. Barbara Bates evaluated continuous waveforms to include possible diseases. She categorized pulse waveform into 5 types shown in 1995 as shown in FIG. 1.4 and Table 1.1 [3].

In order to get more precise information from the pulse wave, researchers take the traditional pulse diagnosis as the reference and map the characteristics of pulse diagnosis with the pattern of waveform [80]. This can be used to detect certain cardiovascular risks and determine the classification. For example, acute anterior myocardial infarction will have a sharp systolic component and very small diastolic component, which suggests poor blood supply based on this pulse wave analysis model.

American Heart Association medical guidelines suggest Framingham heart study as risk source for cardiovascular system evaluation [61]. The Framingham heart study was initialized for studying the correlation between arteriosclerotic heart disease and hypertension in 1950. More than 5000 people were involved in the research [20]. Based on Framingham heart study report, 2223 papers have been published by 2010 [28].

The risk chart from Framingham investigators are used worldwide [18]. The major risks include age, sex, smoking, blood pressure, diabetes, blood lipid, etc. [29]. Based on medical records in database, age, sex, weight, smoking, blood pressure, and diabetes are used to verify the cardiovascular risk evaluation from pulse wave analysis.

TABLE 1.1
Possible diseases which can be diagnosed based on the
different types of cardiovascular pulse shapes [3].

| Pulse type | Physiological cause | Possible disease |
|---|---|---|
| small & | decreased stroke volume | heart failure, hypovolemia, |
| weak | increased peripheral resistance | severe aortic stenosis |
| large & | Increased stroke volume | fever, anaemia, |
| bounding | decreased peripheral resistance | hyperthyroidism, aortic |
| | decreased compliance | regurgitation, bradycardia, |
| | | heart block, atherosclerosis |
| bisferiens | Increased arterial pulse with | aortic regurgitation, aortic |
| | double systolic peak | stenosis and regurgitation, |
| | | hypertropic cardiomyopathy |
| pulsus | pulse amplitude varies from | left ventricular failure |
| alternans | peak to peak, rhythm basically | |
| | regular | |

### 1.4 Pervasive Computing

Pervasive computing is the technology of information and communication that uses miniaturized, embedded, and networked sensors to assist daily lives [41]. As the result of progress in ubiquitous devices, wireless communication, and networks, pervasive computing brings more and more benefits to the health informatics field [60]. Pervasive computing is categorized by the environment while the computer itself is no longer visible. Monitoring and communications can be done under any condition, any place and anytime. It not only means greater availability but also better quality and user acceptance [41].

For example, research shows that the Home Monitoring System can achieve higher quality data than Office/Clinic System [13]. Without anxiety experienced at a clinic, some special effects can be avoided such as White Coat Hypertension. Related home blood pressure testing had been performed for 524 patients from a single general practice with 12 month follow up. 89% of the patients completed the trial which shows high user acceptance. As the result, home blood pressure fallen down 5.2/3.2 mmHg and control rates reached 44.8% from original 29%. The system uses short interval successive home blood pressure readings to give better prediction [13]. In 2009, Kazuo Eguchi suggested that 1 minute successive home blood pressure readings result in a much closer value to the ambulatory blood pressure [22]. Stroke risk reduces by 40% when blood pressure is controlled within recommended levels.

Pervasive Computing is widely applicable to many special fields for cardiac care. It can help physicians recognize 91% of lead related Implantable Cardioverter-Defibrillator (ICD) complications and allow physicians to react quickly [71]. Monitoring has been used for elderly heart failure patients and can achieve a similar result as specialist care. Real time monitoring can be performed with acceptable latency and high fault tolerance. A client side computer can detect cardiac risk with build-in criteria and training data [19].

The Pervasive Computing system is designed to be portable. All sensors can be easily added to or removed from the device so that the system can reach the balance of usability and convenience [67]. It also makes the system widely applicable for different environments other than just home or clinical office. Cardiovascular data can be collected under various circumstances. It provides more valuable data which is helpful to better understand a person's cardiac condition [17].

### Chapter 2

### Subjects and Methods

### 2.1 Overview

The proposed pulse wave analysis system contains following parts:
Finger clip with infra-red sensor
Analog-to-digital (AD) converter and filter
USB interface
Signal analysis software

The infra-red sensor monitors the blood flow at finger, which collects the pulse wave signal. The system uses AD converter to generate digital signal based on the analog pulse wave signal and transmits the pulse wave data to computer through USB interface. Pulse wave data is stored in computer and analyzed using the pulse wave analysis model.

### 2.2 Hardware

The system has the following hardware requirements:
Reliable: the sensor should generate strong consistent signal that represent the pulse pressure. The result should be reproducible under similar conditions.
Portable: data collection will be much more difficult when inviting people to go to a specific location other than meeting them at their own convenience.
Adaptive: the device needs to be adaptive to fit different people for both body size and pulse strength.
Easy to use without professional knowledge: the key reason to use a pulse wave device instead of Electrocardiography and other devices.
Open interface: the system should have an open interface to build analytic modules. Both device and database can be easily accessed through this interface.

The first generation of testing devices uses pressure sensors at the wrist The radial artery has a strong pulse pressure and locates close the skin surface. The signal is convenient and easy to detect. A sample pulse wave from wrist is shown in FIG. 2.1

There are some difficulties for consistency and long term monitoring. Pulse waveforms will change slightly every time depending on how tight the sensor is attached to the wrist and the position where the sensor is placed. It is not comfortable and therefore cannot be used for long periods of time.

In order to make the test more consistent and convenient, this system uses second generation device. It transmits infrared light and is placed in the right index finger. The infra-red sensor, as shown in FIG. 2.2, detects the blood flow at finger and tracks the strength of the blood flow as pulse wave signal. To record a pulse wave, the patients were comfortably rested with the right hand supported. The finger clip was applied to the right index finger. Only the appropriate and stable waveform will be recorded. A sample pulse wave from finger is shown in FIG. 2.3. The basic shapes for pulse wave data collected from wrist and finger are very similar. Infra-red sensor provides precise result that the waveform goes smoothly. The pulse wave data used in this research are collected by the second generation devices. The pulse wave monitoring devices are made by Anhui Huake Electronic Technical Research Institute in China. The device type is HKG-07C with 5V USB power supply. The dimension is 90×50×20 mm for body and 65×25×20 mm for infra-red sensor. The operating condition is 0° C. to 40° C. with relative humidity 15% to 95%.

The infra-red sensor generates two types of infra-red light with wavelength 660 nm and 905 nm. It can also be used for SpO2 monitoring. The sensor fits for both child and adult.

FIG. 2.4 shows the design diagram for AD converter. The signal is filtered by High-pass filtering in FIG. 2.5.

The device has a USB connection to the computer which makes data easily collected. It uses a Silicon Labs CP210x chip to provide UART interface from the USB port. Drivers are provided under multiple Operating Systems such as Windows, Mac OS, Linux, etc. It can transfer data with transmit/receive buffers and modem handshake signals at USB 2.0 full speed. The infra-red sensor at finger clip can monitor the transmittance of the nger and generate byte value according to that. The sampling rate is 200 Hz.

On the computer side, a time serial is collected at the rate of 200 points per second. The calculation for time interval between two points is based on this rate. The program will link all the points as the graph of pulse wave. Similar waveforms with normal components (e.g. systolic components and diastolic components) are the triggers to stop receiving signals and analyze the data. Other medical information is manually entered.

The pulse wave device is ultra-portable. A finger clip can work by itself for pulse wave monitoring. It can be easily integrated into other portable monitoring systems such as the life shirt. A mobile monitoring system can be proposed with this concept.

### 2.3 Software

The system is built on .net framework on a Windows platform.

Multi-threading programming technique has been applied to collect data from the COM port. A driver is responsible for communication with hardware through one of the COM ports. Commands and data are in binary format. Each byte received can be converted into a short value that represents a point on the pulse wave. The value range for pulse wave data is between 0 and 255.

The device works at the frequency of 200 Hz, which means that it returns 200 points per second. Pulse wave is plotted point by point by a high performance computer when it can afford the refresh rate for 200 Hz. There is an adaptive mode for the system to work at half or lower frequency: refreshed for every two or more points. Low performance computers and portable devices can work in this mode. It also helps if the system works with multiple sensors at the same time. Multiple sensors can be used to monitor the time delay for pulse wave at different positions and calculate the Pulse Wave Velocity.

Client side data is stored in an access database which can be easily backed up. Raw pulse wave data is stored in binary format. Factors calculated from the pulse wave are also stored for quick reference.

### 2.4 Error Detection and Correction

The system has several mechanisms to keep the data reliable: ensure the data is collected under stable conditions; have tested value ranges; and establish personal records data for unusual data detection.

Checking the frequency of pulse signal is the first step of quality assurance. The program opens a COM port to accept data at the frequency of 200 Hz.

The stack is limited that might lose data when system is busy. The pulse data is valid only if the signal is collected at a working frequency. In this case, the program should receive 200 points per seconds for the monitoring time period. Patients are required to calm down before taking the test in order to reduce the effects of activities and emotions. Heart rate is a very helpful factor to determine a patients condition. If the heart rate goes higher or change rapidly, the patient might be nervous about testing or a result of recent physical activities. The result is more reliable if the pulse data is taken after the heart rate becomes normal. The program will try to catch the continuous waveforms with similar heart rate (similar number of points per waveform). Most pulse data were collected in the heart rate range 60 to 90 beats per minute.

The valid value range of pulse wave factors can be used for abnormal signal detection. The impact of other systems other than cardiovascular, such as muscle movement could be conspicuous. They can be easily detected with the valid value range. Retaking the test or extending the test for longer time is valid solutions to verify the result.

Comparing history data can find possible errors too. Suspicious records will be reviewed manually and will be retaken if necessary.

### 2.5 Subjects

With informed consent, 607 sets of testing data were collected from 298 subjects in Jinan, Shandong, China. The data collecting phrase started from August 2008 and ended at August 2010. The age of subjects ranged from 1 to 91 years (mean±SD, 53.16±20.85). 132 subjects in control group were chosen randomly from people outside hospital (mean age±SD, 41.38±23.41). The rest records were collected from patients in Department of Cardiology at Shandong Provincial Hospital in China (mean age±SD, 62.52±18.40). There are 389 sets of pulse wave data from 166 people of patient group and 218 sets of pulse wave data from 132 people of control group. Detail statistics of age, weight and blood pressure is shown in Table 2.1.

TABLE 2.1
Age, weight and systolic blood pressure
of patient group and control group.

| | | Num- | | | Systolic |
|---|---|---|---|---|---|
| | | ber of | | | Blood |
| | | sub- | | Weight | Pressure |
| | | jects | Age | (kg) | (mmHg) |
| Patient | Fe- | 61 | 65.35 ± 14.38 | 59.77 ± 8.27 | 119.58 ± 18.78 |
| group | male | | | | |
| | Male | 105 | 60.88 ± 17.65 | 71.93 ± 10.87 | 121.58 ± 20.05 |
| Control | Fe- | 69 | 43.47 ± 27.28 | 49.24 ± 21.63 | 111.58 ± 16.21 |
| Group | male | | | | |
| | Male | 63 | 39.11 ± 22.19 | 65.39 ± 20.06 | 113.26 ± 15.76 |

In order to check pulse wave in different age groups, 25 sets of pulse wave data were collected from infants and children (mean age±SD, 7.84±5.72). Those infants and children are the youngest subjects in this research. Age groups of subjects are shown in FIG. 2.6:

All medical records were collected in order to do research on each risk factor. Risk factor groups and diseases groups were created based on medical records. Subjects with more than 10 years history of continuous smoking are selected into smoking group.

Cardiovascular risk factors and diseases are used to evaluate the patients with medical records. Patients could have more than one disease in Table 2.2.

TABLE 2.2
Age, weight and systolic blood pressure of diseases groups.

| | num- | | | | |
|---|---|---|---|---|---|
| | ber of | Num- | | | Systolic |
| | pulse | ber of | | | Blood |
| | wave | sub- | | Weight | Pressure |
| | data | jects | Age | (kg) | (mmHg) |
| Coro- | 307 | 125 | 60.00 ± 18.05 | 68.10 ± 11.91 | 118.97 ± 19.08 |
| nary | | | | | |
| disease | | | | | |
| Heart | 18 | 5 | 70.60 ± 7.83 | 62.89 ± 9.66 | 106.06 ± 13.22 |
| failure | | | | | |
| Hyper- | 277 | 116 | 59.69 ± 18.34 | 67.59 ± 12.29 | 123.82 ± 18.89 |
| tension | | | | | |
| Dia- | 124 | 54 | 58.91 ± 15.39 | 68.54 ± 13.45 | 124.61 ± 19.00 |
| betes | | | | | |

The cardiovascular risk for subjects is evaluated based on blood pressure, smoking, and diabetes, which are major risk factors from Framingham heart study [18]. Low density lipoprotein and high density lipoprotein are not included in the evaluation because they are not available for all subjects. There are 194 subjects in high risk group and 104 subjects in low risk group. Most patients are in high risk group. Details are shown in Table 2.3:

TABLE 2.3
Age, weight and systolic blood pressure of risk groups.

| | Num- | | | | |
|---|---|---|---|---|---|
| | ber of | Num- | | | Systolic |
| | pulse | ber of | | | Blood |
| | wave | sub- | | Weight | Pressure |
| | data | jects | Age | (kg) | (mmHg) |
| Low | 180 | 104 | 38.71 ± 22.05 | 57.13 ± 21.61 | 110.97 ± 19.18 |
| risk | | | | | |
| High | 427 | 194 | 60.91 ± 18.83 | 67.89 ± 10.75 | 122.07 ± 15.72 |
| risk | | | | | |

### 2.6 Statistics

Associations between two fields were examined by the Pearson product-moment correlation coefficient. The Pearson product-moment correlation is used to evaluate the correlation between two sets of values [65].

The result of correlation is called Pearsons r. It has the value between −1 and 1. Positive result means that two variables increase together. Negative result suggests that one variable increases while another variable decrease. The higher degree of correlation will have either bigger positive result or less negative result [65]. P value is used to show the significance of the correlation. Significance was assigned at P<0.05.

Analysis is performed by Sigmaplot version 12. Data from risk factor groups are imported into separate worksheets in Sigmaplot. The correlation result and graph are generated by integrated functions of Sigmaplot.

### Chapter 3

### Pulse Wave Analysis Model

### 3.1 Model Introduction

There are two major parts in this pulse wave analysis model: factor based analysis and waveform based analysis. They cover the evaluation for cardiovascular functions, general cardiovascular condition, special risks and diseases, and similar cases. The pulse wave analysis model structure is shown as Table 3.1.

Factor based analysis retrieves pulse wave factors that focusing on a part of pulse wave data. It is used to evaluate certain cardiovascular functions. The algorithms have existing standards for diagnosis and less complexity for implementation. For example, stiffness index is used to evaluate the elasticity of blood vessels by calculations with special points of systolic top and diastolic top in pulse wave data.

Waveform based analysis treats the pulse wave data as a whole. It provides estimation for general cardiovascular conditions and can be used for classification and similar case detection. For example, dynamic time warping can classify the pulse wave data by calculating the differences between testing data and sample data.

TABLE 3.1
Model structure for pulse wave analysis.

| | | advantages | algorithm | Usage |
|---|---|---|---|---|
| Pulse | Factor | less complex, | Stiffness | arterial |
| Wave | Based | easy to | Index | stiffness |
| Analy- | Analy- | implement, | Cardiac | cardiac |
| sis | sis | existing standard | output | function |
| | wave- | overall | histogram | waveform shape |
| | form | estimation, | | (smooth or |
| | based | classification | | sharp) |
| | analy- | | bispectrum | waveform type |
| | sis | | estimation | |
| | | | Wavelet and | detect abnormal |
| | | | Morlet Wavelet | component |
| | | | Weighted | classification |
| | | | Dynamic time | and similar |
| | | | Warping | waveform |

Stiffness index and cardiac output have existing algorithms and evaluation criterions. The proposed pulse wave analysis model has adjustment on stiffness index to make it more sensitive to arterial stiffness.

Weighted dynamic time warping is specialized algorithm for pulse wave analysis which is first introduced in this model. It can achieve better accuracy than original algorithm.

The proposed model with factor based analysis and waveform based analysis is adaptive for cardiovascular evaluation: both general conditions and specific risks.

### 3.2 Factor Based Analysis

Pulse wave factors can be classified into two categories: basic factors and derivative factors. Basic factors are retrieved from pulse wave data directly while derivative factors are calculated from basic factors. Stroke volume is calculated from pulse wave data and cardiac output can be calculated by stroke volume and pulse rate. Therefore, Cardiac output is a derivative factor of stroke volume.

### 3.2.1 Stiffness Index

As shown in FIG. 3.1, the first part of the waveform (systolic component) is the result of pressure transmissions along a direct path from the aortic root to the wrist. The second part (diastolic component) is caused by the pressure transmitted from the ventricle along the aorta to the lower body. The time interval between the diastolic component and the systolic component depends upon the Pulse Wave Velocity of the pressure waves within the aorta and large arteries which is related to arterial stiffness. The stiffness index is an estimation of the Pulse Wave Velocity about arterial stiffness and is obtained from subject height (h) divided by the time between the systolic and diastolic peaks of the pulse wave contour. The height of the diastolic component of the pulse wave relates to the amount of pressure wave reflection.

$$S I = \frac{h}{Δ {T}_{DVP}} \qquad ( 3.1 )$$

As observation of testing result, there is little difference for stiffness index when the pulse rate changes because it is calculated by the time interval between systole and diastole. Testing subjects get a little higher stiffness index after exercise. Another observation is that younger people with high pulse rate can get a relative high score than older people with slow pulse rate. The average stiffness index is 8.59 for patients in age range 10 to 20 which is much higher than patients in age range 20 to 30. The calculation can be modified based on this situation. Adjustment with pulse rate is applied to the stiffness index calculation.

$$Adjusted S I = \frac{S I \times 60}{Pulse Rate} \qquad ( 3.2 )$$

This is not an ideal adjustment since pulse rate only has limited effects on the stiffness index.

### 3.2.2 Cardiac Output

The pulse wave method for calculating cardiac output is done based on the theory of elastic cavity [46] [44]. The calculation used in this pulse wave analysis model is based on the method from Bing Nan Li in 2005 [44].

Blood flow continuous equation:

$${Q}_{in} = {Q}_{out} + \frac{d V}{d {t}_{1}} ( t ∈ {T}_{1} ) \\ {Q}_{out} + \frac{d V}{d {t}_{2}} = 0 ( t ∈ {T}_{2} ) \qquad ( 3.3 )$$

where Qin is the volume of blood that pumped into the artery and Qout is the volume of blood flowing into the vein. T1 is systolic period and T2 is diastolic period.

Equation between pressure remainder and blood flow:

$${Q}_{out} = \frac{p - {p}_{v}}{R} \qquad ( 3.4 )$$

where p is the arterial pressure, pᵥ is the venous pressure, and R is the peripheral resistance of cardiovascular system.

Arterial Pressure Volume Equation

$$A C = \frac{d V}{d p} \qquad ( 3.5 )$$

where AC is a constant that depends on the arterial compliance.

Based on the Equations 3.3, 3.4, and 3.5, the analytic equation of elastic cavity can be calculated:

$${Q}_{in} = A C \frac{d p}{d {t}_{1}} + \frac{P - {P}_{v}}{R} ( t ∈ {T}_{1} ) \\ A C \frac{d p}{d {t}_{2}} + \frac{P - {P}_{v}}{R} = 0 ( t ∈ {T}_{2} ) \qquad ( 3.6 )$$

Computing the integral of the equation:

$${S}_{v} = A C ( {P}_{S}^{*} - {P}_{d} ) + \frac{{A}_{S}}{R} \\ A C ( {P}_{d} - {P}_{S}^{*} ) + \frac{{A}_{d}}{R} = 0 \qquad ( 3.7 )$$

where Sᵥ is the stroke volume during a heartbeat. A_{S}, A_{d}, P*_{S}, and P_{d} are shown in FIG. 3.2.
The stroke volume can be calculated by:

$${S}_{v} = \frac{0.28}{{k}^{2}} T ( {P}_{s} - {P}_{d} ) \qquad ( 3.8 )$$

where k is auxiliary blood pressure index defined as:

$$k = \frac{{\int }_{0}^{T} \overline{P} d t}{T ( {P}_{s} - {P}_{d} )} = \frac{\overline{P} - {P}_{d}}{{P}_{s} - {P}_{d}} \qquad ( 3.9 )$$

Cardiac output can be calculated by:

CO=Sᵥ×Pulse rate (3.10)

### 3.3 Waveform Based Analysis

Waveform analysis could be done in two ways: single waveform analysis and continuous waveform analysis. Single waveform is a complete waveform that records pulse wave data from the start point of systolic component to the start point of the next systolic component. Single waveform is used as basic unit for waveform analysis. Continuous waveforms usually contain different number of single waveforms in different time intervals. They are important for detection of some special diseases such as Arrhythmia.

### 3.3.1 Waveform Slope Histogram

Slope is a geometric concept showing the relationship between a line and the axis. Slope of the line containing two adjacent pulse wave points indicates the variation of signal at the specific time point. The histogram of waveform slope can be used to determine the basic shape of the waveform

S(k)=P(k)−P(k−1) (3.11)

where S(k) is slope at point k and P(k) is the pulse wave value of k.

The categories of histogram for data to fill are called bins [15]. This analysis model uses maximum 10 bins for all histograms.

The major part of a histogram will have a balance distribution around 0 when the slope has fewer changes.

The histogram is very useful to real time waveform estimation.

### 3.3.2 Bispectrum Estimation

Fourier transform is the foundation of many signal processing algorithms including Bispectrum estimation [57]. Bispectrum, which is also called third order spectra, is a special case of higher order spectra. It can retrieve features of deviations from Gaussianness and estimate the phase of non-Gaussian parametric signals [56]. Typical pulse waves from low risk subjects and high risk subjects have been used to extract the bispectrum features of sub-health condition [37].

Higher order spectra contain deviations from Gaussianness and nonlinear information [56]. It is cumulant spectra defined as

$$\begin{matrix}{{{c}_{{k}_{1} … {k}_{n}} ( - j )}^{r} {=}^{Δ} \frac{{\partial }^{r} ln Φ ( {ω}_{1} , {ω}_{2} , … , {ω}_{n} )}{\partial {ω}^{{k}_{1}} {ω}^{{k}_{2}} … {ω}^{{k}_{n}}} \vert}_{{ω}_{1} = {ω}_{2} = … = {ω}_{n} = 0} \\ where & ( 3.12 ) \\ Φ ( {ω}_{1} , {ω}_{2} , … {ω}_{n} ) = E \{ exp j ( {ω}_{1} {x}_{1} + … + {ω}_{n} {x}_{n} ) \} & ( 3.13 )\end{matrix}$$

is the joint characteristic function of real random variables x₁, x₁, . . . xₙ.

The N^{th} order spectrum C (ω₁, ω₂, . . . ωₙ) of process {X(k)} can be expressed as the Fourier transform of its N^{th} order cumulant sequence C_{N}(T₁, T₂, . . . T_{N-1})

$$C ( {ω}_{1} , {ω}_{2} , … {ω}_{N - 1} ) = {\sum }_{{τ}_{1} = - ∞}^{+ ∞} … {\sum }_{{τ}_{N - 1} = - ∞}^{+ ∞} {C}_{N} ( {τ}_{1} , {τ}_{2} , … {τ}_{N - 1} ) \cdot exp \{ - j ( {ω}_{1} {τ}_{1} + … + {ω}_{n - 1} {τ}_{n - 1} ) \} \qquad ( 3.14 )$$

It is Power Spectrum when N=2. Bispectrum is also called 3^{rd} order spectrum. It can be expressed as

$${B}_{x} ( {ω}_{1} , {ω}_{2} ) = {C}_{3 x} ( {ω}_{1} , {ω}_{2} ) = {\sum }_{{τ}_{1} = - ∞}^{+ ∞} {\sum }_{{τ}_{2} = - ∞}^{+ ∞} {C}_{N} ( {τ}_{1} , {τ}_{2} ) \cdot exp \{ - j ( {ω}_{1} {τ}_{1} + {ω}_{2} {τ}_{2} ) \} \qquad ( 3.15 )$$

The direct method for estimation groups the pulse data into k sets with M values in each set.

The total value of pulse data N=KM [57]. Then process the pulse data:

P^{l}(x)=p(x)− (̅k̅)̅ (3.16)

where x belongs to set k.

The discrete Fourier transform coefficients can be calculated by [57]

$${Y}^{( i )} ( λ ) = \frac{1}{M} {\sum }_{k = 0}^{M - 1} {x}^{( i )} ( k ) exp ( - j2π k λ M ) \qquad ( 3.17 )$$

where x⁽ⁱ⁾(k) is the i^{th} set of data and λ=0, 1, . . . , M/2. Then bispectrum of discrete Fourier transform coefficient is

$$\hat{{b}_{i}} ( {λ}_{1} , {λ}_{2} ) = \frac{1}{{Δ}_{0}^{2}} {\sum }_{{k}_{1} = - {L}_{1}}^{{L}_{1}} {\sum }_{{k}_{2} = - {L}_{1}}^{{L}_{1}} {Y}^{( i )} ( {λ}_{1} + {k}_{1} ) {Y}^{( i )} ( {λ}_{2} + {k}_{2} ) {Y}^{( i ) +} \cdot ( {λ}_{1} + {k}_{1} + {λ}_{2} + {k}_{2} ) \qquad ( 3.18 )$$

The bispectrum estimation of pulse data is the average value of the k coefficients [57]:

$$\hat{{C}_{3}^{x}} ( {ω}_{1} , \vert {ω}_{2} ) = \frac{1}{k} {\int }_{i = 1}^{k} \hat{{b}_{i}} ( {ω}_{1} , {ω}_{2} ) \\ where \\ {ω}_{1} = ( \frac{2 π {f}_{s}}{{N}_{0}} ) ( {λ}_{1} ) and {ω}_{2} = ( \frac{2 π {f}_{s}}{{N}_{0}} ) ( {λ}_{2} ) \qquad ( 3.19 )$$

High risk pulse waveform and low risk pulse waveform usually have different value distribution for bispectrum estimation, which suggests that bispectrum is suitable for risk detection.

3.3.3 Continuous Wavelet transform and Morlet Wavelet analysis

Wavelet transform is well known for localized variations of power analysis. It uses the time and frequency domains together to describe the variability. The algorithm can extract information from many kinds of data including audio and images especially in geophysics fields. It has been used to analyze tropical convection [81], the El NioSouthern Oscillation [36], atmospheric cold fronts [30], central England temperature [2], the dispersion of ocean waves [52], and wave growth and breaking [47].

There are some research that apply Wavelet algorithm to Pulse wave-form. A wavelet-based cascaded adaptive filter was used to resolve baseline drift problems in pulse waveform [85]. Some pulse wave monitoring devices cannot generate a constant signal and the baseline is changed among pulse waveforms. Lisheng Xu introduced wavelet-based cascaded adaptive filter to adjust baseline drift in 2005 [85].

Wavelet analysis can use both single waveform and continuous waveforms because it has frequency component on the time domain. Dividing a continuous time signal into wavelets is called Continuous Wavelet Transform. Regarding to the original pulse wave data f(x), the transform is defined as

$$\begin{matrix}{W}_{ψ} ( s , τ ) = {\int }_{- ∞}^{∞} f ( x ) {ψ}_{s , τ} ( x ) d x \\ where & ( 3.20 ) \\ {ψ}_{s , t} ( x ) = \frac{1}{\sqrt{s}} ( \frac{x - τ}{s} ) & ( 3.21 )\end{matrix}$$

and S is the continuous scale parameter and is continuous translation parameter [34]. The scale parameter defines the degree of signal compression. Smaller scale parameter will generate more detail in result. (x) is called square integrable function. The converse function can be expressed as

$$f ( x ) = \frac{1}{{C}_{ψ}} {\int }_{0}^{∞} {\int }_{- ∞}^{∞} {W}_{ψ} ( s , τ ) \frac{{ψ}_{s , τ} ( x )}{{s}^{2}} d τ d s \qquad ( 3.22 )$$

Morlet wavelet convert the wavelet transform as [45]

$$ψ ( x ) = exp ( - \frac{{β}^{2} {x}^{2}}{2} ) cos ( π x ) \qquad ( 3.23 )$$

It can also be expressed with scale parameter and translation parameter

$${ψ}_{s , τ} ( x ) = exp [ \frac{{{β}^{2} ( x - τ )}^{2}}{{s}^{2}} ] cos [ \frac{π ( x - τ )}{s} ] \qquad ( 3.24 )$$

The Morlet wavelet can be used to replace the original wavelet function in continuous wavelet transform.

FIG. 3.3 and FIG. 3.4 show the difference between continuous wavelet transform and continuous wavelet transform with Morlet wavelength. The result for continuous wavelet transform is a two-dimensional matrix. The features can be extracted by finding the low value paths. The combination of continuous wavelet transform and continuous Morlet wavelet transform not only shows abnormal components but also evaluates the shape of pulse waveform.

### 3.3.4 Weighted Dynamic Time Warping

One of the most fundamental concepts in the nonlinear pattern recognition is time-warping. The dynamic time warping proposed by Sakoe and Chiba is one of the most versatile algorithms in speech recognition [69].

Initially, most dynamic time warping applications were in the field speech recognition [69]. It achieves a higher recognition rate with lower cost than most other algorithms. Medical data has been analyzed with dynamic time warping recently. Electrocardiogram is one of the most common signals in health care environment, so most research focus on electrocardiogram signal analysis [38].

Dynamic Time Warping was applied to electrocardiogram segmentation because segmenting the electrocardiogram automatically is the foundation for abnormal conduction detection and all analysis tasks. Dynamic time warping based single lead method achieves a smaller mean error with higher standard deviation than the two-lead Lagunas method [79].

Modified Dynamic Time Warping was used in pulse waveform recognition by Lu Wang in 2004 [79]. Derivative Dynamic Time Warping is proposed to decrease false rejection ratio and false acceptance ratio. However, it did not provide a solution to adjust the dynamic time warping result with clinical significance of pulse wave.

Since pulse data is two dimensional time serial data, the mining techniques for time serial data can be applied. The waveforms can be categorized based on the similarity between testing waveform and well classified sample waveforms. Because the waveforms have same structure: taller systolic component with lower diastolic component following, the similarity calculation can achieve high accuracy. It can be measured by the total distance of corresponding points between sample waveform and testing waveform warping.

FIG. 3.5 (a) and (d) are two waveforms to compare. FIG. 3.5 (b) point to point differenced. FIG. 3.5 (c) shows the point based warping result based on dynamic time warping.

A sample waveform is denoted {xₜ(j), 1≦j≦J} and an unknown frame of the signal {x(i), 1≦i≦l}. The time warping provides a mapping between the time indices i and j such that a time registration between the waveforms is obtained. The mapping can be represented by a sequence of points c=(i,j), between i and j as [69] [38].

M={c(k),1≦k≦K} (3.2)

where c(k)=(i(k), j(k)) and {x(i), 1≦i≦l} is testing data, {xₜ(j); 1≦j≦J} is the template data.
Warping function finds the minimal distance between two sets of data:

d(c(k))=d(i(k),j(k))=∥x(i(k))−xₜ(j(k))∥₂ (3.26)

The smaller value of d is, the higher the similarity between x(i) and xₜ(j) detected.

The optimal path minimize the accumulated distance D_{T}:

$${D}_{T} = {min}_{\{ M \}} {\sum }_{k = 1}^{K} d ( c ( k ) ) w ( k ) \qquad ( 3.27 )$$

where w(k) is a non-negative weighting coefficient.

To find the optimal path, the following calculation needs to be performed:

D(c(k))=d(c(k))+min(D(c(k−1))) (3.28)

where D(c(k)) represents the minimal accumulated distance.

There are two restrictions for warping pulse wave:
Monotonic condition: i(k−1)≦i(k) and j(k−1)≦j(k)
Continuity Condition: i(k)−i(k−1)≦1 and j(k)−j(k−1)≦1

The symmetric dynamic warping equation with slope of 1 is:

$$D ( c ( k ) ) = d ( c ( k ) ) + min ( \begin{matrix}D ( i ( k - 1 ) , j ( k - 2 ) ) + 2 d ( i ( k ) , j ( k - 1 ) ) \\ D ( i ( k - 1 ) , j ( k - 1 ) ) + 2 d ( c ( k ) ) \\ D ( i ( k - 2 ) , j ( k - 1 ) ) + 2 d ( i ( k - 1 ) , j ( k ) )\end{matrix} ) \qquad ( 3.29 )$$

The optimal accumulated distance is normalized by (i+j) for symmetric form.

Each pulse wave category has its own clinical features. For example, the shape of systolic component is more important for heart failure patients and the shape for diastolic component should be emphasized for patients with coronary artery disease. Evaluating these components separately for different pulse wave categories is the solution to improve the classification result.

Weight vectors are included in Dynamic Time Warping to improve the result with medical significance. A weight vector has same length as a corresponding sample pulse wave data. Weights are assigned by the clinical significance of its pulse wave category. If sample waveform belongs to coronary artery disease category, weights for diastolic component have bigger values. Heart failure samples will have higher weights for systolic component. The distance from a point in sample waveform to testing waveform is defined as the actual distance times corresponding weight.

The weighted dynamic time warping algorithm is the base of this pulse wave analysis model. It is involved for risk detection, disease detection, and similar case reference.

### Chapter 4

### Result and Discussion

The result is discussed in two steps: algorithm features and case study. Algorithm features are derived from both factor based analysis and waveform based analysis. Detail result is discussed in case study.

### 4.1 Factor Based Analysis

Stiffness index is the major pulse wave factor in this analysis model. The correlation between stiffness index and cardiovascular condition is verified among different risk and disease groups.

The mean value for stiffness index of all 607 records from Jinan is 8.110 (standard deviation 1.729). Age, hypertension, smoking, and diabetes are commonly used risk factors for cardiovascular disease [5]. They are used to verify the relationship between cardiovascular risks and pulse wave analysis.

The smoking group consists 40 sets of pulse wave data collected from 18 subjects. Smoking group has much higher stiffness index than the average level. The mean value is 10.039 with standard deviation 2.587. Diabetes group contains 124 records from 54 people. Stiffness index in Diabetes group is also higher than the average level: mean value 9.975, standard deviation 2.174. 389 pulse wave data in patients group are collected from the Department of Cardiology at Shandong Provincial Hospital and 307 of them have coronary artery diseases which are caused by arterial stiffness.

TABLE 4.1
Stiffness index of different groups.

| | Number | Mean value | |
|---|---|---|---|
| | of pulse | of Stiffness | Standard |
| Group | wave data | Index | deviation |
| Over all | 607 | 8.110 | 1.729 |
| Patient | 389 | 8.332 | 1.806 |
| Control | 218 | 7.710 | 1.502 |
| No risk | 78 | 7.558 | 1.751 |
| Coronary | 307 | 8.587 | 1.997 |
| Hypertension | 277 | 8.226 | 1.956 |
| Diabetes | 124 | 9.975 | 2.174 |
| Smoking | 40 | 10.039 | 2.587 |

Table 4.1 shows that smoking group and diabetes group have much higher stiffness index than other groups, while control group has the lowest stiffness index. However, coronary artery disease group has lower stiffness index than diabetes group and smoking group. Two facts could be the reason for this situation: patients with coronary artery disease were taking medicines regarding to their cardiovascular system; people in diabetes group and smoking group could have coronary artery disease as well. Medical treatment can improve cardiovascular condition for patients. People in risk group without medical treatment and people with multiple risks will likely have higher stiffness index.

Other basic records, such as weight, do not have clear correlation with stiffness index.

Age is a risk factor that affects everyone. The mean age for all pulse wave data is 56.055 with standard deviation 20.851. The Pearson correlation r=0.190 and P=0.00000382. Stiffness index is positively correlated with age since r is positive and P less than 0.050. But the correlation is not significant.

As shown in FIG. 4.2, stiffness index also has no significant correlation with Systolic Blood Pressure: Pearsons r=0.192, P=0.00000299.

Pearsons r is 0.572 with P 3.141E-012 for no risk group and 0.167 for the patient group. Stiffness index has much higher correlation with age in the no risk group. Age is the major risk factor for people without other risks. However, when people have other risk factors such as smoking and diabetes, SI no longer has a significant correlation with age. It also indicates that SI is sensitive to cardiovascular diseases and risk factors. People who have cardiovascular diseases or risks will have higher stiffness index. This makes SI a perceptible indicator in diagnosing arterial stiffness.

SI can be affected by the cardiac condition as described before. The adjusted SI can only rectify influence of heart rate in a certain level. Other abnormal cardiac conditions, such as heart failure, will disturb the pulse waveform in different ways. A basic adjustment of cardiac condition will make SI more sensitive.

The testing results based on age are shown in FIG. 4.4, which indicate that the adjusted stiffness index is more sensitive than stiffness index. Pearsons r for adjusted stiffness index is 0.352 with P 1.961E-018. The correlation between stiffness index and Systolic Blood Pressure is increased to 0.197 after adjustment. The adjustment has positive effects on the stiffness index that the adjusted stiffness index is more sensitive to the cardiovascular risk factors.

When grouping pulse wave data with medical records of coronary artery disease together, the adjusted stiffness index has a higher value 8.578±0.116 than the stiffness index 8.332±1.806. On the other hand, pulse wave data in control group of coronary artery diseases have a lower adjusted stiffness index 7.605±1.950 when compared to stiffness index 7.710±1.502.

The adjustment on stiffness index can achieve higher accuracy for risk factor detection.

No risk group has higher cardiac output than control group, which is mixed with risk and no risk people. Coronary artery diseases group has the lowest cardiac output. It suggests that people without cardiovascular risks have better cardiac function.

TABLE 4.2
Cardiac output of different groups.

| | Number | Mean value | |
|---|---|---|---|
| | of pulse | of Cardiac | Standard |
| Group | wave data | Output | deviation |
| Over all | 607 | 5.646 | 1.868 |
| Patient | 389 | 5.408 | 1.396 |
| Control | 218 | 6.105 | 1.482 |
| No risk | 78 | 6.540 | 1.328 |
| Coronary | 307 | 5.436 | 1.381 |
| Hypertension | 277 | 5.563 | 1.458 |
| Diabetes | 124 | 5.780 | 1.508 |

Cardiac output is not strongly correlated to age, weight, and systolic blood pressure. It shows the working status of the heart while SI shows the degree of arterial stiffness. Patients group have lower cardiac output than control group But there is no significant correlation between stiffness index and cardiac output. Therefore, cardiac output is a good complement of stiffness index for analyzing cardiovascular condition.

### 4.2 Waveform Analysis

### 4.2.1 Waveform Slope Histogram

Different shape of waveforms affects the distribution of histogram of waveform slopes.

Histogram of a typical normal waveform has a negative slope allocated in a small range and positive slopes distributed in a larger range. There is no majority column having much higher value than any other columns. The histogram has near normal distribution: the closer to zero, the higher values.

The peak value appears at the negative side.

High risk pulse waveforms usually lose normal components. The waveform slope has fewer changes than normal waveform. It could have the following changes:
When the waveform lose diastolic component, one column at the range of −1 to −2 has much higher value than all other columns.
When patient has abnormal cardiac function, the columns in range 0 to 2 have higher values than other columns.
The combination of abnormal cardiac function and arterial stiffness will result in the non-normal distribution in histogram.

The histogram provides an overview for the shape of pulse waveform with less computational complexity.

### 4.2.2 Bispectrum Estimation

The bispectrum estimation has a more centralized distribution in normal pulse waveform as shown in FIG. 4.6. The low frequency parts have more outstanding values: most high value part allocated within the square area from −0.05 to 0.05. The high values are usually allocated in 4 parts. Peak values are much higher than the bispectrum estimation of high risk waveforms.

The bispectrum estimation has more distributed structure for pulse waveform with risks. In FIG. 4.7, the low frequency parts have more outstanding values, but the high values are extended to more areas. The high values are usually allocated in 4 parts, which could be merged together. Peak values are much lower than the bispectrum estimation of normal waveforms.

Arrhythmia is a common abnormal electrical activity in cardiovascular system. The heart rate might go too fast or too slow which will cause the waveforms change shape among continuous pulse waves. This feature can be captured in both time domain and frequency domain. The basic feature in time domain is pulse wave duration variance among continuous pulses exceeding the average level. The incomplete waveforms and merged waveforms often result in the pulse detection fails which is also a sign of arrhythmia.

The bispectrum estimation for Arrhythmia is very similar to high risk waveforms discussed before except a sharp edge could be detected at all high value areas.

FIG. 4.8 shows that Arrhythmia cause the second pulse arrives in advanced while the first pulse waveform is not complete yet.

### 4.2.3 Continuous Wavelet Transform and Morlet Wavelet

Continuous Wavelet transform for regular waveform has two significant local minimum paths representing systolic component and diastolic component. It does not provide too much information other than the components.

The effects of pulse wave components can be identified from wavelet transform. The beginning low value path is caused by the systolic component. The systolic pressure and time duration can be evaluated by the first low value path. The second small low value path represents diastolic component. Morlet wavelet shows derivatives at bottom scales: high values for positive and low values for negative. Systolic component and diastolic component can be located by changes at bottom line. The normal pulse wave increases rapidly at the beginning and decreases slowly after the peak value. The Morlet wavelet transform has several approximate straight low value paths representing the basic shape.

Waveforms with abnormal components can be easily identified by comparing to the wavelet transform of normal waveform. For example, FIG. 4.9 shows a waveform with normal systolic component, diastolic component and an abnormal ending. It will have relatively normal result for Dynamic Time Warping or other similar based algorithms. Classification can achieve higher accuracy when including the abnormal component detection from wavelet transform.

Abnormal component detection has been used in waveform classification with Dynamic Time warping. When the waveforms of the category have similar shape as normal waveform, classification with dynamic time warping has high error rate.

The wavelet transform of high risk pulse waveform shows that the diastolic component is missing. The first low value path appears later because the pulse has a weak systolic component. The low value paths in Morlet wavelet transform is crooked which suggests that pulse wave has an abnormal shape.

### 4.2.4 Weighted Dynamic Time Warping

FIG. 4.12 shows a matrix of point to point distances between two pulse wave forms. The matrix is the intermediate product of dynamic time warping. It can help to understand the result of dynamic time warping.

The matrix stores the distances for all points between two pulse waveforms. For each point in testing waveform, there is a minimum distance to sample waveform in the matrix. The dynamic time warping distance is the sum of those minimum distances. The line connecting all minimum distances is the minimum path of the dynamic time warping. Warping waveforms can be generated based on the minimum path mapping. The result of warping process are illustrated in FIG. 4.13.

A typical waveform for people in the age group 20 to 30 has well defined systolic component and diastolic component. FIG. 4.14 shows an example. The signal line goes smoothly from the beginning to the end of the waveform. The data reading increases significantly in the systolic part because of a powerful heartbeat. It decreases rapidly to the time point that pulse reflection arrived. The diastolic component locates in the middle area of the descending part of the waveform. It suggests that a certain illness condition exits when the diastolic component appears too early or too late.

For people from 30 to 50, the pulse waveform remains regular shape except the diastolic component becomes weak. FIG. 4.15 shows one of samples in this age group. The diastolic component might lose the local maximum value but it still can be easily distinguished from the descending part of the waveform.

For elderly people, the diastolic component is weaker, sometimes even difficult to find. As previously mentioned in the stiffness index calculation, the diastolic component is allocated at the peak value of the first derivative when missing the local maximum value. Waveforms are very different among people in different health conditions. Some distinct shapes or features can be used to detect possible cardiovascular risks.

In addition to the cardiovascular risk classification, the waveform similarity can be used as major standard to the detection of certain cardiovascular diseases.

### 4.3 Classification

Some testing were done on classification with proposed model to support future research. Risk classification and similar cased reference are based on the weighted dynamic time warping. Disease detection also includes other features in Table 5.1.

### 4.3.1 Classification for Cardiovascular Risk

Cardiovascular risks were evaluated for 607 sets of pulse wave data. Age, blood pressure, smoking, diabetes, hypertension are used as evaluation criteria based on Framingham heart study [61]. 427 sets of pulse wave data are classified in a high risk group, which is 64% of all pulse wave data. 180 sets of pulse wave data belong to the low risk group.

The risk of pulse wave data is evaluated by weighted dynamic time warping. Three high risk waveforms and three low risk waveforms were selected as the sample data for pulse wave categorization. The selection is based on the waveform classifications from Bates and Dawber [3] [21].

The average value for distance between testing waveform and sample waveforms is 63673. The average value for minimum distance, which is used to determine the category, is 30660. If the minimum distance between testing data and sample waveforms is greater than 50000, then the testing data is not similar to any of the sample waveforms; if the minimum distance is less than 10000, then the testing data and selected sample waveform are similar. 73 sets of data do not have a good match in samples and 225 sets of test data have a close match to the samples. More samples will increase matcher in the classification. There are 96 misclassified sets of records and the overall accuracy for classification is 84.2%.

TABLE 4.3
Classification result using dynamic time warping.

| | | Number of | | |
|---|---|---|---|---|
| | Number of | waveforms | Accuracy | False |
| Risk | waveforms | predict to | for risk | rejection |
| group | in category | be high risk | detection | rate |
| Low risk 1 | 102 | 27 | 74.5% | 26.7% |
| Low risk 2 | 42 | 5 | 88.1% | |
| Low risk 3 | 36 | 16 | 55.5% | |
| High risk 1 | 175 | 163 | 93.1% | 11.2% |
| High risk 2 | 137 | 120 | 87.6% | |
| High risk 3 | 115 | 96 | 83.5% | |

High risk data has the biggest properties in the result because most data were collected from patients in the department of cardiovascular medicine. Healthy people in the control group contribute greatly to the low risk category.

### 4.3.2 Disease Detection

Disease detection is based on the study of waveform. Four typical waveforms with acute anterior myocardial infarction, old myocardial infarction, ventricular aneurysm and dilated cardiomyopathy are used as the sample. Acute anterior myocardial infarction and old myocardial infarction are selected because they have significant features for the shape of the waveform. Ventricular aneurysm and dilated cardiomyopathy are selected because their waveforms have similar shape to normal waveforms. Weighted dynamic time warping is the basic standard for disease detection. The sample with minimum dynamic time warping has the same category with testing waveform. The result is shown in Table 4.4 Result for disease detection with weighted dynamic time warping (similarity level: distance<15000).

TABLE 4.4
Result for disease detection with weighted dynamic
time warping (similarity level: distance <15000).

| | Acute | | | |
|---|---|---|---|---|
| | anterior | Old | | Dilated |
| | myocardial | myocardial | Ventricular | cardio- |
| | infarction | infarction | aneurysm | myopathy |
| # of waveform | 72 | 51 | 13 | 37 |
| that have | | | | |
| related medical | | | | |
| history | | | | |
| # of waveforms | 73 | 37 | 60 | 29 |
| detected | | | | |
| # of detected | 45 | 33 | 8 | 17 |
| waveforms that | | | | |
| have related | | | | |
| medical history | | | | |
| Accuracy | 61.6% | 89.2% | 13.3% | 58.6% |
| False rejection | 37.5% | 35.3% | 38.5% | 54.1% |
| rate | | | | |

Medical history review indicates a positive result. Accuracy for ventricular aneurysm is much higher than others since the waveform of ventricular aneurysm is very similar to the normal waveform. Testing data are easily categorized in mistake. With higher similarity level (distance<5000), 23 sets of data is detected and 6 of them have related medical history. Abnormal component detection can reduce error rate too. Abnormal components are detected from 10 waveforms and 6 of them have related disease in medical records. The false rejection rate for dilated cardiomyopathy is higher than others. Most of missing waveforms have less dynamic time warping distance to normal waveform. Based on these two observations, the more similar to normal form, the higher error rate and false rejection rate will occur. Waveform of acute anterior myocardial infarction has very different shape to normal waveform. Some children with congenital heart diseases have similar waveforms due to the quick heart rate. Age check additional accuracy for classification: 49 waveforms are classified into this category and 45 of them have related medical records. The error rate in this category is reduced to 8.2%.

### 4.3.3 Similar Cases Reference

The dynamic time warping distance is calculated among all 607 records. 5 records with lowest dynamic time warping distance are selected as similar cases for each record. Evaluation has been done with coronary artery disease, diabetes, and hypertension. Table 4.5 Disease detection with similar cases reference shows the number of records that and the same disease in the similar cases. For example, there are 307 records having coronary artery disease in pulse wave database. 167 of them and the same disease in the most similar case. 265 of them have the coronary artery disease reference in top 5 similar cases.

TABLE 4.5
Disease detection with similar cases reference.

| Number of | | | |
|---|---|---|---|
| similar cases | Coronary | | |
| involved | disease | Diabetes | Hypertension |
| 1 | 167 | 22 | 149 |
| 2 | 244 | 37 | 191 |
| 3 | 244 | 51 | 212 |
| 4 | 257 | 64 | 227 |
| 5 | 265 | 79 | 235 |
| Actual records | 307 | 124 | 277 |
| with the disease | | | |
| Detection rate | 86.3% | 63.7% | 84.8% |

The detection only includes 5 similar cases which is less than 1% of all records. The detection rate is over 80% for coronary artery disease and hypertension and 63.7% for diabetes.

It not only detects possible risks but also provides medical records for references. It can assist health professionals to take the next steps, schedule additional test and avoid possible mistakes or delays.

Most testing subjects were patients from the hospital which is the shortcoming of the classification test. Expanding the test subject base to include more non-patients should be considered in future research to provide better test sample.

### Chapter 5

### Case Study

Cardiovascular diseases have special effects on pulse wave. Common features can be derived by study of similar cases. This chapter focuses on several major cardiovascular diseases from the department of cardiovascular medicine and discusses the features of those diseases in pulse wave analysis.

### 5.1 Acute Anterior Myocardial Infarction

The pulse wave in FIG. 5.1 was taken from a male patient at department of cardiology. He had a history of myocardial infarction for 8 years and came to the clinic again because of angina pectoris. His cardiac function was rated as New York Heart Association classification grade IV and had to stay in bed and rest.

The waveform is typical with poor cardiac function. The systolic part is very sharp and narrow that suggests very low cardiac output. The diastolic component is lost because of a weak pulse. Blood vessel condition is not measurable because the myocardial infarction is in an acute stage.

The histogram is balance distributed based on 0. It is the result of poor cardiac function and losing diastolic component. The blood pressure quickly dropped after the systolic component. The positive value of slope at increasing part and the negative value of slope at decreasing part are relatively stable. There is no significant change for waveform of those two parts. The high value areas of bispectrum estimation are extended and merged. It does not have normal 4-parts distribution for high values. Abnormal components can be found in continuous wavelet transform. The low value paths in Morlet wavelet transform are crooked. The characteristics of this pulse wave can be summarized as:
Low pulse pressure
Low cardiac output
Waveform around the baseline is longer than normal
Sharp and narrow systolic component
No diastolic component
High detection rate with dynamic time warping
High value areas in bispectrum estimation are extended and merged
Abnormal component exists in wavelet transform
Crooked low value path in Morlet wavelet transform

### 5.2 Old Myocardial Infarction

The pulse wave in FIG. 5.4 is collected from a patient with old myocardial infarction and degenerative valvular disease. He has chest distress and ictal thoracalgia for eighteen years. Gasping happened for the recent 6 months and the pain increased in intensity for the last 3 months. The patient also has mitral regurgitation and tricuspid regurgitation that make him difficult to finish some daily activities. His cardiac function is rated New York Heart Association classification grade IV.

The waveform has strong systolic component and visible diastolic component. The systolic part becomes broader than usual which might because of the compensatory blood supply after myocardial infarction. The waveform has multiple peak values after systolic top which is the result of old myocardial infarction and degenerative valvular disease.

The histogram has majority bin at small negative values and more bins at positive values. The bins of positive big values show that the patient has normal cardiac function in compensatory stage. High value areas in bispectrum estimation are merged. There is no clear low value path representing the diastolic component in wavelet transform. Morlet wavelet transform has normal low value paths.

With review of similar waveforms and medical history, waveforms in this category have:
A broader systolic component
The diastolic component could have different shape depending on the arteries condition.
The cardiac output usually has normal relative high values.
There is majority bin in histogram
High value areas in bispectrum are merged together.
There is no clear low value path representing the diastolic component.

### 5.3 Ventricular Aneurysm

The pulse wave in FIG. 5.7 belongs to a 57 years old male patient. Coronary angiography shows that stenosis at left anterior descending artery that reduces 40%-50% of the arterys capacity. The first diagonal branch and left circumflex also have stenosis. Ventricular aneurysm occupies 30% chambers of the heart.

The systolic part of waveform does not have very clear features. The diastolic component goes in a vertical direction longer than normal waveform. A little uplift could be observed at the end of diastolic component.

The histogram is very similar as the histogram of normal waveform. The high value areas are extended but they are still distributed in 4 parts. Abnormal component can be detected in wavelet transform. There is an additional low value path that covers more area than the low value path representing diastolic component. Morlet wavelet transform does not show any abnormal feature.

There are eight patients with Ventricular aneurysm in the pulse database and 6 of them have a pulse wave that belongs to this category.
Major significance in diastolic part, gives more weight when calculating dynamic time warping distance
Wavelet transform shows abnormal component

### 5.4 Dilated Cardiomyopathy

A fifteen year old male patient took the pulse wave test after admission in hospital had dilated cardiomyopathy. He had palpitation for eight year history and had oliguresis, edema of lower extremity for recent 3 months. He had fast heart rate which could reach 140/min. The heart border expanded to left and the pulse was weak. Cardiac ultrasonic shows that left ventricle had spherical expansion. The interventricular septum and ventricular wall were thin. The cardiac output and cardiac index decreased. His pulse wave is shown in FIG. 5.10

Pulse waveform of dilated cardiomyopathy usually have deep and significant diastolic component. More values fill into the bins from 0 to 4. The high value areas in bispectrum are expanded. Wavelet transform shows a longer and wider low value path representing the diastolic component. Low value paths in Morlet wavelet have normal shape.

FIG. 5.12 shows similar waveforms detected by dynamic time warping. The patients for those two waveform also have dilated cardiomyopathy.

Dilated cardiomyopathy has the following features:
Patients could be young—less than 20 years of age.
Slow slope changes at both systolic top and diastolic top.
Normal or low stiffness index.
Abnormal cardiac output.
High pulse rate before medical treatment.
High value areas are expanded in bispectrum estimation

### 5.5 Coronary Artery Spasm

The patient is a 31 years old male without a record of cardiovascular disease before admission. He had acute chest pain after continuous smoking. Coronary angiography shows that coronary has stenosis about 40%.

The major characteristic of this pulse waveform in FIG. 5.13 is an abnormal systolic component. The patient is just 31 years old without arterial stiffness. The waveform has phanic diastolic component and stiffness index is 7.20. The cardiac output is 3.12 which is less than normal range. The systolic pressure is also less than normal waveforms.

The major feature for waveform analysis of coronary artery spasm is weak systolic component. Bispectrum estimation shows expended high value areas. There is no meaningful feature can be retrieved from the histogram and wavelet transform.

The following features need to be included for coronary artery spasm detection:
Young patients
Normal stiffness index
Low cardiac output
Low systolic pressure
Give more weight for systolic component for waveform similarity analysis
Slow changing for slopes at both systolic component and diastolic component
High value areas expended for bispectrum estimation

### 5.6 Diastolic Hypertension

FIG. 5.15 shows the pulse wave from a 46 year old male with diastolic hypertension. He had headaches and dizziness one year before admission. The symptoms relieved after taking rest. The systolic blood pressure and diastolic blood pressure are 140 mmHg/105 mmHg. He was diagnosed as diastolic hypertension after admission. Diastolic hypertension often combined with other cardiovascular diseases such as coronary artery diseases. Missing diastolic component in this waveform suggests that the patient has serious arterial stiffness. The bottom-line for the waveform is near 80. It is much higher than normal waveforms which usually have bottom-line below 40. Morlet wavelet transform has distorted low value path. It is also caused by the abnormal shape of waveform.

The pulse wave analysis for this case includes:
High diastolic pressure
Low pulse pressure. The base line for this pulse wave is over 70 while the maximum value is under 170.
High stiffness index. The stiffness index of this patient is 8.73 while high end of stiffness index is 9 in age 40 to 50.
Histogram has majority bin at positive side.
Abnormal component can be detected in wavelet transform.
Low value paths are distorted in Morlet wavelet transform

### 5.7 Heart Failure

The patient is a 72 year old male with 30 year history of hypertension. He also has renal failure for 20 years. The Admission reason is palpitation, dyspnea, tachycardia, and cannot lie down. The blood pressure is 140/110 mmHg. He was diagnosed as heart failure with high output.

The stiffness index is 12.19 for pulse wave analysis and cardiac output is 6.52. He has unstable pulse rate of 103 per minute.

The major feature for waveform analysis of heart failure is sharp systolic component with high pulse rate. The cardiac output is much lower than normal. The waveforms within the same pulse wave data may very different because the heart failure often come with arrhythmia.

The pulse wave for heart failure has the following features:
Often has other diseases: myocardial infarction, arrhythmia
Could have normal waveform at paracmasis
Pulse rate is high and unstable in acute stage.
The systolic pressure and pulse pressure are not stable.
The patient could have normal or low cardiac output (depends on the heart failure type).

### 5.8 Arrhythmia

The 62 year old female had undergone a radical mastectomy 9 years ago. She has no history of cardiovascular disease. She was admitted because of a heart. Premature was recorded by Electrocardiogram.

Premature is featured by early coming of next waveform. It can be detected by the distances among continuous systolic components. If one distance is shorter and the next distance becomes longer, it usually suggests a premature.

The pulse wave of sinoatrial block is from a 55 year old male patient. He has short period dizziness for over 3 years and admitted for sinoatrial block type II. His heart rate reached a high of 140 and a low of 39.

Sinoatrial block is on the opposite side of premature: a systolic component comes much later than normal. It can be detected by find a much longer distance between two adjacent systolic components.

Arrhythmia can be easily detected by pulse wave:
Abnormal pulse rate for people with pulse rate over 100 or less than 60
Pulse rate variance. Arrhythmia can be detected by time differences among continuous pulse waves. It usually has over 50% time variance
Abnormal component could be detected in wavelet transform
Time intervals between waveforms are very important for arrhythmia detection
Dynamic time warping distances among waveforms within same pulse wave data have big differences

Features of the cases can be summarized in the Table 5.1

TABLE 5.1
Pulse wave features for disease detection

| | Acute | | | | | | | |
|---|---|---|---|---|---|---|---|---|
| | anterior | Old | | Dilated | Coronary | | | |
| | myocardial | myocardial | Ventricular | cardio- | artery | Diastolic | Heart | |
| | infarction | infarction | aneurysm | myopathy | spasm | hypertension | failure | Arrhythmia |
| Age | elder people | elder | elder people | young | Young | elder people | — | — |
| | | people | | | people | | people | |
| Blood | — | — | — | — | low | high | low | — |
| pressure | | | | | | | | |
| Stiffness | High | high | High or | — | — | high | high | — |
| Index | | | normal | | | | | |
| Cardiac | Low | — | Low or | low | low | | low | Low or |
| output | | | normal | | | | | normal |
| Histogram | Near Normal | Majority | — | — | — | — | Abnormal | — |
| | distribution | column | | | | | distribution | |
| | | exist | | | | | | |
| Bispectrum | High | High | Sharp | Sharp | High | — | Sharp edge | — |
| | value area | value | edge | edge | value | | for high | |
| | merged | area | for high | for high | area | | value area | |
| | | merged | value | value | extended | | | |
| | | | area | area | | | | |
| Hilbert- | | | — | — | — | Component | Component | Less variance |
| Huang | | | | | | have less | have less | |
| | | | | | | variance | variance | |
| Wavelet | Abnormal | Diastolic | Abnormal | — | — | Possible | Possible | — |
| | component | component | component | | | abnormal | abnormal | |
| | detected | missing | detected | | | component | component | |
| Dynamic | High detect | High detection | Low detection | Low | High | High detection | High detection | High detection |
| Time | rate | rate | rate | detection | detection | rate | rate | rate |
| Warping | | | | rate | rate | | | |

### Chapter 6

### Conclusion and Future Work

### 6.1 Conclusion

The goal of this thesis is to create an appropriate model for cardiovascular health evaluation with pulse wave data. The research is proposed based on some facts and other research on pulse wave analysis:
Pulse has been treated as an important life signal and has been used to detect health condition for more than two thousand years.
Pulse signal is widely used for surgery, medicine and other fields.
Many factors can be retrieved from the pulse dada such as pulse wave velocity, stiffness index, and cardiac output.
The relationships between some cardiovascular diseases and pulse wave factors have been proved to be trustable. For example, the stiffness index is very sensitive to arterial stiffness.
The thesis proposes the model and points out the strength and weakness of each algorithm in the model. Several pattern recognition techniques have been applied to the pulse wave data in order to make the model more adaptive.
Pulse wave factors have a positive relationship with cardiovascular conditions, but only some of them can achieve high accuracy. They need to be evaluated together to achieve more reliable result.
Bispectrum is good at waveform type validation and special waveform detection.
Wavelet and Morlet wavelet are used for continuous wavelet transform. They can identify abnormal components and evaluate waveform shape.
Weighted dynamic time warping emphasizes the similarity of waveforms. It can be used for waveform classification and disease detection when the sample waveform data are well defined. It can also provide similar cases for decision support.

Based on this result, the analysis techniques have strengths in different areas. Pulse wave factors have good detection rate for cardiovascular risks. Waveform analysis is more suitable for over all cardiovascular health evaluation and pulse wave classification. The combination of both strategies increased the reliability of pulse wave analysis.

This thesis also evaluated the pulse wave analysis model by taking tests of cardiovascular risk classification, diseases detection and similar cases reference. Most pulse wave research focus on arterial stiffness and related area such as early detection of vascular disease [17]. Few of them discussed the relationship between pulse waveform and potential diseases [3]. The model proposed in this thesis provides a practical way to find the clinical meanings of pulse waveforms. Large amount of clinical pulse wave data were analyzed with the model and features were extracted for specific cardiovascular diseases. This is the major contribution which can benefit future research.

### REFERENCES

[1] Asmar, Roland, Athanase Benetos, Jirar Topouchian, Pierre Laurent, Bruno Pannier, Anne-Marie Brisac, Ralph Target And Bernard I. Levy. Assessment of Arterial Distensibility by Automatic Pulse Wave Velocity Measurement. Hypertension, n. 26, p. 485, 1995.
[2] Baliunas, Sallie, Peter Frick, Dmitry Sokoloff, and Willie Soon. Time scales and trends in the central England temperature data. Geophysical Research Letters, v. 24, n. 11, p. 1351-1354, June 1997.
[3] Bates, Barbara. A Guide to Physical Examination, 6th edition. Philadelphia, USA.: J.B. Lippingcott Company, 1995.
[4] Bedford, D. Evan. The ancient art of feeling the pulse. British Heart Journal, v. 13, p. 423-437, October 1951.
[5] Benjamin, Emelia J., Philip A. Wolf, Ralph B. D'Agostino, Halit Silbershatz, William B. Karma and Daniel Levy. Impact of Atrial Fibrillation on the Risk of Death The Framingham Heart Study. Circulation, v. 98, p. 946-952, 1998.
[6] Berndt, Donald J., and James Clifford. Using Dynamic Time Warping to Find Patterns in Time Series. workshop on knowledge discovery in databases. [S.1.]: AAAI. 1994. p. 359-370.
[7] Berton, Christine and Bernard Cholley. Equipment review: New techniques for cardiac output measurement oesophageal Doppler, Fick principle using carbon dioxide, and pulse contour analysis. Critical Care, v. 6, p. 216-220, 2002.
[8] Blacher, Jacques, Alain P. Guerin, Bruno Pannier, Sylvain J. Marchais, and Grard M. London. Aortic Pulse Wave Velocity as a Marker of Cardiovascular Risk in Hypertensive Patients. Hypertension, v. 33, p. 1111-1117, 1999.
[9] Blacher, Jacques, Roland Asmar, Saliha Djane, Grard M. London, and Michel E. Safar. Arterial Calcifications, Arterial Stiffness, and Cardiovascular Risk in End-Stage Renal Disease. Hypertension, v. 38, p. 938-942, 2001.
[10] Brandfonbrener, Martin, Milton Landownd, and Nathan W. Shock. Changes in Cardiac Output with Age. Circulation, 12 1955: 557-566.
[11] Braunwald, Eugene, Zipes Douglass P. Libby Petter. Heart disease: a textbook of cardiovascular medicine. 6th. ed. [S.1.]: WB Saunders Co, 2001.
[12] Brigham, E. O., R. E. Morrow. The fast Fourier transform. Spectrum, IEEE, v. 4, n. 12, p. 63-70, 1967.
[13] Byrnes, Patrick D, Geoffrey K Mitchell, Margaret V Crawford, and Colleen McGoldrick. A cardiovascular risk clinic using. Australian Family Physician, 38, 2009.
[14] Cain, M E., D. Ambos, F X. Witkowski, and B E. Sobel. Fast-Fourier transform analysis of signal-averaged electrocardiograms for identification of patients prone to sustained ventricular tachycardia. Circulation, v. 69, p. 711-720, 1984.
[15] Chapelle, Olivier, Patrick Haffner, and Vladimir N. Vapnik. Support vector machines for histogram-based image classification. Neural Networks, IEEE Transactions on, v. 10, n. 5, p. 1055-1064, September 1999.
[16] Cholley, Bernard P., Sanjeev G. Shroff, Joanne Sandelski, Claudia Korcarz, Beth A. Balasia, Shelly Jain, David S. Berger, Michael B. Murphy, Richard H. Marcus, Roberto M. Lang. Differential effects of chronic oral antihypertensive therapies on systemic arterial circulation and ventricular energetics in African-American patients. Circulation, v. 91, p. 1052-1062, 1995.
[17] Cohn, Jay N., Stanley Finkelstein, Gary McVeigh, Dennis Morgan, Lisa LeMay, Jennifer Robinson, and James Mock. Noninvasive Pulse Wave Analysis for the Early Detection of Vascular Disease. Hypertension; v. 26, p. 503-508, 1995.
[18] Conroy, R. M., K. Pyrl, A. P. Fitzgerald, S. Sans, A. Menotti, G. De Backer, D. De Bacquer, P. Ducimetire, P. Jousilahti, U. Keil, I. Njlstad, R. G. Oganov, T. Thomsen, H. Tunstall-Ped.oe, A. Tverdal, H. Wedel, P. Whincup, L. Wilhelmsen, and I. M. Graham. Estimation of ten-year risk of fatal cardiovascular disease in Europe: the SCORE project. European Heart Journal, v. 24, n. 11, p. 987-1003, 2003.
[19] Dar, Owais, Jillian Riley, Callum Chapman, Simon W. Dubrey, Stephen. Morris, Stuart D. Rosen, Michael Roughton and Martin R. Cowie. A randomized trial of home telemonitoring in a typical elderly heart failure population in North West London: results of the Home-HF study. European journal of heart failure, 11, 2009.
[20] Dawber, Thomas R., William B Kannel, Nicholas Revotskic, and Abraham Kagan. The Epidemiology of Coronary Heart Disease The Framingham Enquiry. Proceedings of the Royal Society of Medicine, v. 55, n. 4, p. 265-271, April 1962.
[21] Dawber, T R, H E Jr Thomas, and P M. McNamara. Characteristics of the dicrotic notch of the arterial pulse wave in coronary heart disease. Angiology, v. 24, n. 4, p. 244-255, April 1973.
[22] Eguchi, Kazuo, Sujith Kuruvilla, Gbenga Ogedegbe, William Gerin, Joseph E Schwartz, and Thomas G Pickering. What is the optimal interval between successive home blood. Journal of Hypertension, 27, 2009.
[23] Erlanger, J., and D. R. Hooker. Johns Hopi. Hosp. Rep, p. 357, 1904.
[24] Fan, Zhaopeng, Gong Zhang, Simon Liao. Clinical Analysis for Cardiovascular Disease by Calculating Stiffness Index, Cardiac Output from Pulse Wave IEEE Canadian Conference on Electrical and Computer Engineering 2009
[25] Fan, Zhaopeng, Gong Zhang, Simon Liao. Cardiovascular Remote Monitoring System with Electronic Health Record The 33rd Conference of the Canadian Medical and Biological Engineering Society, 2010.
[26] Fan, Zhaopeng, Gong Zhang, Simon Liao. Pulse wave analysis. Advanced Biomedical Engineering, p. 21-40, 2011.
[27] Felbinger, T W., D A. Reuter, H K. Eltzschig, J. Bayerlein, and A E. Goetz. Cardiac index measurements during rapid preload changes: a comparison of pulmonary artery thermodilution with arterial pulse contour analysis. J Clin Anesth, v. 17, p. 241-248, 2005.
[28] FRAMINGHAM Heart Study. Bibliography. Framingham heart study, 2011. Disponivel em: <http://www.framinghamheartstudy.org/biblio/index.html>. Acesso em: 26 jul. 2011.
[29] FRAMINGHAM Heart Study. Framingham Chart.
[30] Carnage, N.; Blumen, W. Comparative analysis of lowlevel cold fronts: Wavelet, Fourier, and empirical orthogonal function decompositions. Monthly weather review, v. 121, p. 2867-2878, 1993.
[31] Ganz, William, Roberto Donoso, Harold S. Marcus, James S. Forrester, and Harold J. C. Swan. A new technique for measurement of cardiac output by thermodilution in man. The American Journal of Cardiology, v. 27, n. 4, p. 392-396, 1970.
[32] Ge, J, A Jeremias, A Rupp, M Abels, D Baumgart, F Liu, M Haude, G Grge, C Von Birgelen, S Sack and R. Erbel. New signs characteristic of myocardial bridging demonstrated by intracoronary ultrasound and Doppler. European Heart Journal, v. 20, n. 23, p. 1707-1716, 1999.
[33] Gillum, Richard F., Diane M. Makuc, and Jacob J. Feldman. Pulse rate, coronary heart disease, and death: The NHANES I Epidemiologic Follow-up Study. American Heart Journal, v. 121, n. i, p. 172-177, 1991.
[34] Gonzalez, Rafael C., and Richard E. Woods. Digital Image Processing. 3rd. ed. New Jersey: Prentice-Hall, Inc., 2008.
[35] Green, Jerry Franklin. Mechanical Concepts in Cardiovascular and Pulmonary Physiology. Philadelphia, Pa., USA.: Lea & Febiger, 1984.
[36] Gu, Daifeng, and Philander H. Secular Changes of Annual and Interannual Variability in the Tropics during the Past Century. Journal of Climate, v. 8, n. 4, p. 864-876, 1995.
[37] Hong, W-X, Z Zhang, Jing, and G-F Jing. Nonparametric bispectrum estimation for pulse signals of sub-health. Journal of Yanshan University, v. 33, n. 3, p. 232-236, 2009.
[38] Huang, Bin and W. Kinsner. ECG Frame classification using dynamic time warping. Electrical and Computer Engineering, 2002. IEEE CCECE 2002. Canadian Conference. 2002. p. 1105-1110.
[39] Jansen, E. W., E. C. Cheriex, I. Kastenlein, C. Borst, P. J. Slootweg, and J. F. Hitchcock. Epicardial cryosurgery as used in ablation of accessory atrioventricular pathways: effect on valvular function in the dog. European Journal of Cardio-Thoracic Surgery, v. 4, p. 445-450, 1990.
[40] Kangasniemi, K, and H Opas. Suornalainen lkrikeskus. Toinen painos. WSOY, Porvoo: Toinen painos, 1997.
[41] Khlera, Andreas, and Lorenz Erdmannb. Expected Environmental Impacts of Pervasive Computing. Human and Ecological Risk Assessment: An International Journal, v. 10, n. 5, p. 831-852, August 2010.
[42] Kozo, Hirata, Kawakami Masanobu, and O'Rourke Michael F. Pulse Wave Analysis and Pulse Wave Velocity: A Review of Blood Pressure Interpretation 100 Years After Korotkov. Circulation journal, v. 70, n. 10, p. 1231-1239, 2006.
[43] Langewouters, G. J., K. H. Wesseling, and W. J. A. Goedhard. The static elastic properties of 45 human thoracic and 20 abdominal aortas in vitro and the parameters of a new model. Journal of Biomechanics, v. 17, n. 6, p. 425435, 1984.
[44] Li, Bing Nan, Ming-Chui Doug, Val Mang, and Mak Peng Un. A novel intelligent sphygmogram analyzer for health monitoring of cardiovascular system. Expert Systems with Applications, v. 28, n. 4, p. 693-700, 2005.
[45] Lin, Jing, and Liangsheng Qu. Feature extraction based on Morlet wavelet and its application for mechanical fault diagnosis. Journal of Sound and Vibration, v. 234, n. 1, p. 135-148, jun. 2000.
[46] Liu, Z. R., and X. X. LI. The theory of elastic cavity and its application in the analysis of cardiovascular system. Science Press, Beijing, 1987.
[47] Liu. Paul C. Wavelet Spectrum Analysis and Ocean Wind Waves. New York: Academic Press, 1994.
[48] Mahomed, F A. The physiological and clinical use of the sphygmograph. Medical Times Gazette, p. 6264, 1872.
[49] Mahomed, F A. The aetiology of brights disease and the prealbuminuric stage. Med Chir Trans, v. 57, p. 197-228, 1874.
[50] Mahomed, F A. On the sphygmographic evidence of arterio-capillary fibrosis. Trans Path Soc, v. 28, p. 394-397, 1877.
[51] McEniery, Carmel M., Yasmin, Ian R. Hall, Ahmad Qasem, Ian B. Wilkinson, and John R. Cockcroft. Normal Vascular Aging: Differential Effects on Wave Reflection and Aortic Pulse Wave Velocity. Journal of the American college of Cardiology, p. 1753-1760, 2005.
[52] Meyers, S. D., B. G. Kelly, and J. J. O'brien. An introduction to wavelet analysis in oceanography and meteorology: With application to the dispersion of Yanai waves. Monthly weather review, v. 121, p. 2858-2866, 1993.
[53] Millasseau, Sandrine C., Franck G. Guigui, Ronan P. Kelly, Krishna Prasad, John R. Cockcroft, James M. Ritter, and Philip J. Chowienczyk Noninvasive Assessment of the Digital Volume Pulse. Hypertension, v. 36, p. 952-956, 2000.
[54] Millasseau, S. C., R. P. Kelly, J. M. Ritter, and P. j. Chowenczyk. Determination of age-related increases in large artery stiffness by digital pulse contour analysis. Clinical Science, v. 103, p. 371-377, 2002.
[55] Mitchell, Gary F., Helen Parise, Emelia J. Benjamin, Martin G. Larson, Michelle J. Keyes, Joseph A. Vita, Ramachandran S. Vasan, and Daniel Levy. Changes in Arterial Stiffness and Wave Reflection With Advancing Age in Healthy Men and Women. Hypertension, v. 43, p. 1239-1245, 2004.
[56] Nikias, C. L., and M. R. Raghuveer. Bispectrum estimation: A digital signal processing framework. Proceedings of the IEEE, v. 75, n. 7, p. 869-891, jul. 1987.
[57] Nikias, C. L., and J. M. Mendel. Signal processing with higher-order spectra. Signal Processing Magazine, IEEE, v. 10, n. 3, p. 10-37, jul. 1993.
[58] ORourke, MF, and G. Mancia. Arterial stiffness. J Hypertension, v. 17, p. 1-4, 1999.
[59] ORourke, M., A. Pauca, and X-J. Jiang. Pulse wave analysis. Br J Pharmacol, v. 51, p. 507-522, 2001.
[60] Orwat, Carsten, Andreas Graefe, and Timm Faulwasser. Towards pervasive computing in health care A literature review. BMC Medical Informatics and Decision Making, v. 8, n. 26, p. 26, June 2008.
[61] Pearson Thomas A., Steven N. Blair, Stephen R. Daniels, Robert H. Eckel, Joan NI. Fair, Stephen P. Fortmann, Barry A. Franklin, Larry B. Goldstein, Philip Greenland, Scott M. Grundy, Yuling Hong, Nancy Houston Miller, Ronald M. Lauer, Ira S. Ockene, Ralph L. Sacco, James F. Sallis Jr, Sidney C. Smith Jr, Neil J. Stone, and Kathryn A. Taubert. AHA Guidelines for Primary Prevention of Cardiovascular Disease and Stroke: 2002 Update. Circulation, v. 106, p. 388-391, 2002.
[62] Persell, Stephen D, Alexis P Dunne, Donald M Lloyd-Jones, and David NV Baker. Electronic Health Record-Based Cardiac Risk Assessment and Identification of Unmet Preventive Needs. Medical Care, 47, 2009.
[63] Petra, Michael A., Sherman G Sorensen, Tuhin K. Chaudhuri, Paul Rosen, and Robert A. O'Rourke. Attenuation correction of equilibrium radionuclide angiography for noninvasive quantitation of cardiac output and ventricular volumes. American Heart Journal, v. 107, n. 6, p. 1221-1228, 1984.
[64] Postel-Vinay, M C. Growth hormone- and prolactin-binding proteins: soluble forms of receptors. Horm Res, v. 45, p. 178181, 1996.
[65] Rodgers, Joseph Lee, and W. Alan Nicewander. Thirteen Ways to Look at the Correlation Coefficient. The American Statistician, v. 42, n. 1, p. 59-66, Feburary 1998.
[66] Rdig, G., C. Prasser, C. Keyl, A. Liebold, and J. Hobbhahn. Continuous cardiac output measurement: pulse contour analysis vs thermodilution technique in cardiac surgical patients. Br J Anaesth, v. 82, p. 525-530, 1999.
[67] Saha, D., and A. Mukherjee. Pervasive computing: a paradigm for the 21st century. Computer, v. 36, n. 3, p. 25-31, March 2003.
[68] Sakka, S. G., C. C. Rhl, U. J. Pfeiffer, R. Beale, A. McLuckie, K. Reinhart and A. Meier-Hellmann. Assessment of cardiac preload and extravascular lung water by single transpulmonary thermodilution. Intensive Care Medicine, v. 26, p. 180-187, 2000.
[69] Sakoe, H., and S. Chiba. Dynamic Programming Optimization for Spoken Word Recongition. IEEE Transactions on Signal Processing, v. 26, p. 43-49, 1978.
[70] Sherwood, Andrew, Michael T. Allen, Jochen Fahrenberg, Robert M. Kelsey, William R. Lovallo, and Lorenz J. P. van Doornen. Methodological guidelines for impedance cardiography. Psychophysiology, v. 27, p. 1-23, January 1990.
[71] Spencker, Sebastian, Nalan Coban, Lydia Koch, Alexander Schirdewan, and Dirk Muller. Potential role of home monitoring to reduce inappropriate shocks in implantable cardioverter-defibrillator patients due to lead failure. Europace, 11, 2009.
[72] Spodick, D H, and V Q Lance. Noninvasive stress testing. Methodology for elimination of the phonocardiogram. Circulation, v. 53, p. 673-676, 1976.
[73] Sutton-Tyrrell, Kim, Anne Newman, Eleanor M. Simonsick, Richard Havlik, Marco Pallor, Edward Lakatta, Harold Spurgeon, and Peter Vaitkevicius Aortic Stiffness Is Associated With Visceral Adiposity in Older Adults Enrolled in the Study of Health, Aging, and Body Coinposition. Hypertension, v. 38, p. 429-433, 2001.
[74] Sutton-Tyrrell, Kim, Samer S. Najjar, Robert M. Boudreau, Lakshmi Venkitachalam, Varant Kupelian, Eleanor M. Simonsick, Richard Havlik, Edward G. Lakatta, Harold Spurgeon, Stephen Kritcheysky, Marco Pahor, Douglas Bauer, and Anne Newman. Elevated Aortic Pulse Wave Velocity, a Marker of Arterial Stiffness, Predicts Cardiovascular Events in Well-Functioning Older Adults. Circulation, v. 111, p. 3384-3390, 2005.
[75] Timothy, S M., E S Barbara, L. Joseph, and Jr Izzo. Validity and Reliability of Diastolic Pulse Contour Analysis (Windkessel Model) in Humans. Hypertension, v. 39, p. 963-968, 2002.
[76] Torrence, Christopher, and Gilbert P. Compo. A Practical Guide to Wavelet Analysis. Bulletin of the American Meteorological Society, v. 79, p. 61, January 1998.
[77] Vullings, H., M. Verhaegen, and H. Verbruggen. Automated ECG segmentation with dynamic time warping. 20th Ann. Int. Conf Hong Kong: IEEE Engineering in Medicine and Biology Soc. 1998. p. 163-166.
[78] Wang, Shuhe. Mai Jing, 280.
[79] Wang, Lu, Kuanquan, Wang, and Lisheng Xu. Recognizing wrist pulse waveforms with improved dynamic time warping algorithm. the Third International Conference on Machine Learning and Cybernetics. Shanghai: [s.n.]. 2004.
[80] Wang, Huiyan, and Yiyu Cheng. A quantitative system for pulse diagnosis in Traditional Chinese Medicine. Engineering in Medicine and Biology Society, p. 5676-5679, January 2006.
[81] Weng, Hengyi, and K.-M. Lau. Wavelets, Period Doubling, and Time-Frequency Localization with Application to Organization of Convection over the Tropical Western Pacific. Journal of Atmospheric Sciences, v. 51, n. 17, p. 2523-2541, 1994.
[82] Wilkinson, Tan B., Ian R. Hall, Helen MacCallum, Isla S. Mackenzie, Cannel M. McEniery, Bart J. van der Arend, Yae-Eun Shu, Laura S. MacKay, David J. Webb, and John R. Cockcroft. Pulse-Wave Analysis—Clinical Evaluation of a Noninvasive, Widely Applicable Method for Assessing Endothelial Function. Arteriosclerosis, Thrombosis, and Vascular Biology, v. 22, p. 147-152, 2002.
[83] WebMD, Webster's New World Medical Dictionary. 3rd. ed. New York: Webster's New World, 2008.
[84] WHO. Cardiovascular diseases (CVDs) Fact sheets. World Health Organization, September 2011. Disponivel em: http://www.who.int/mediacentre/factsheets/fs317/en/index.html. Acesso em: 23 Sep. 2011.
[85] Xu, Lisheng, David Zhang, and Kuanquan Wang. Wavelet-Based Cascaded Adaptive Filter for Removing Baseline Drift in Pulse Waveforms. Biomedical Engineering, v. 52, n. 11, p. 1973-1975, 2005.
[86] Yusuf, Salim, Srinath Reddy, Stephanie unpuu, and Sonia Anand. Global Burden of Cardiovascular Diseases Part I: General Considerations, the Epidemiologic Transition, Risk Factors, and Impact of Urbanization. Circulation, n. 104, p. 2746-2753, 2001.
[87] Zhang, Gong, Xiaoling Kong, and Simon Liao. Pulse wave analysis for cardiovascular information monitoring in patients with chronic heart failure: effects of COQ10 treatment. Bio-engineering. Montreal: [s.n.]. 2008.
[88] Zureik, M, A Benetos, C Neukirch, D Courbon, K Bean, F Thomas, and P Ducimetire. Reduced Pulmonary Function Is Associated with Central Arterial Stiffness in Men. American journal of respiratory and critical care medicine, n. 12, p. 2181-2185164, 2001.

To elaborate on the discussion in Sections 3.3.4 and 4.2.4, the weighting coefficient in Weighted Dynamic Time Warping is used to amplify the effect for the most significant or meaningful part of the waveform (i.e. the part of the waveform that is of interest). It is not a constant set of values, but rather, is a set of values just for one pulse wave data and just for a specific research purpose. For example, when studying the cardiac function, the systolic component may be amplified while the diastolic component may be amplified when studying arterial function.

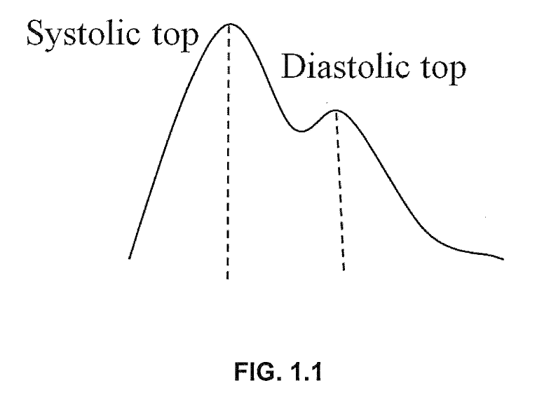

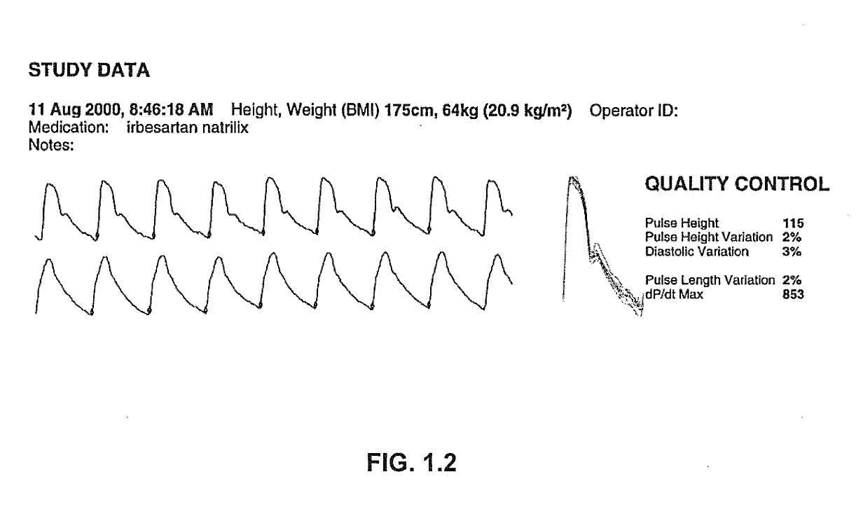

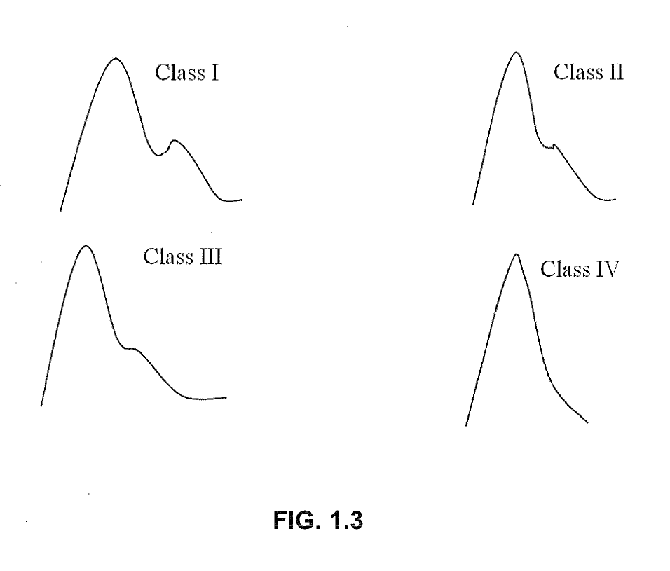

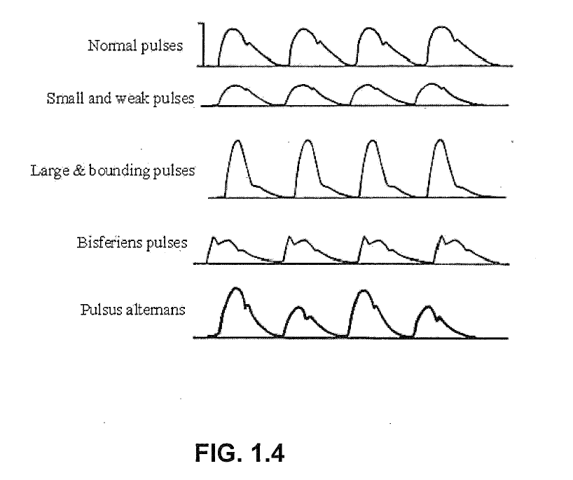

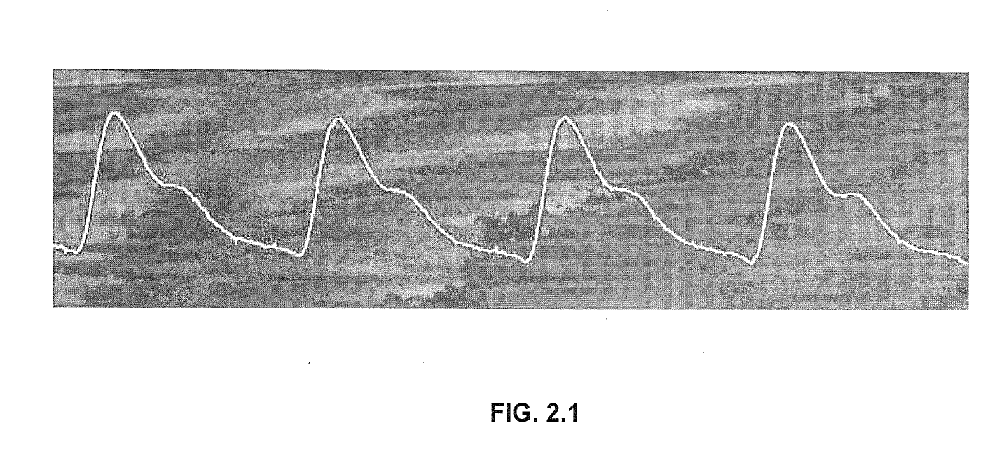

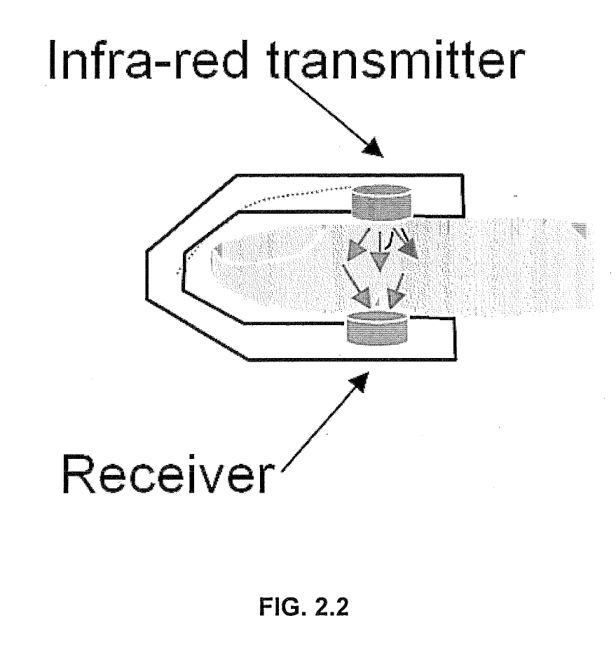

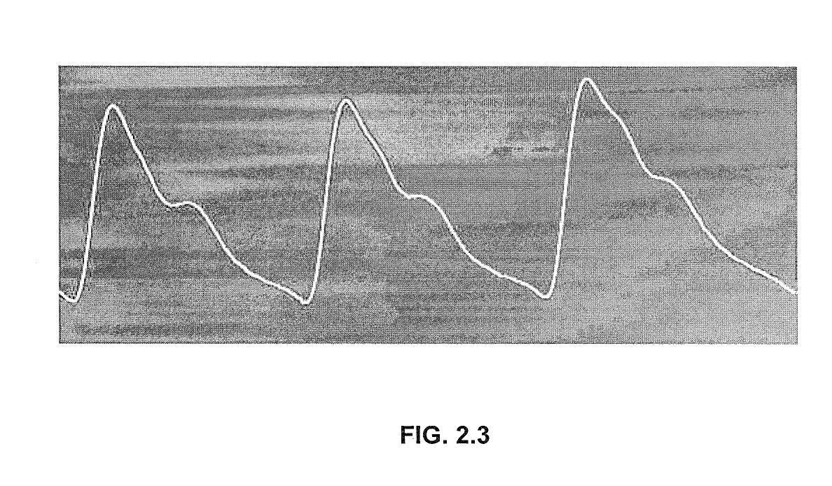

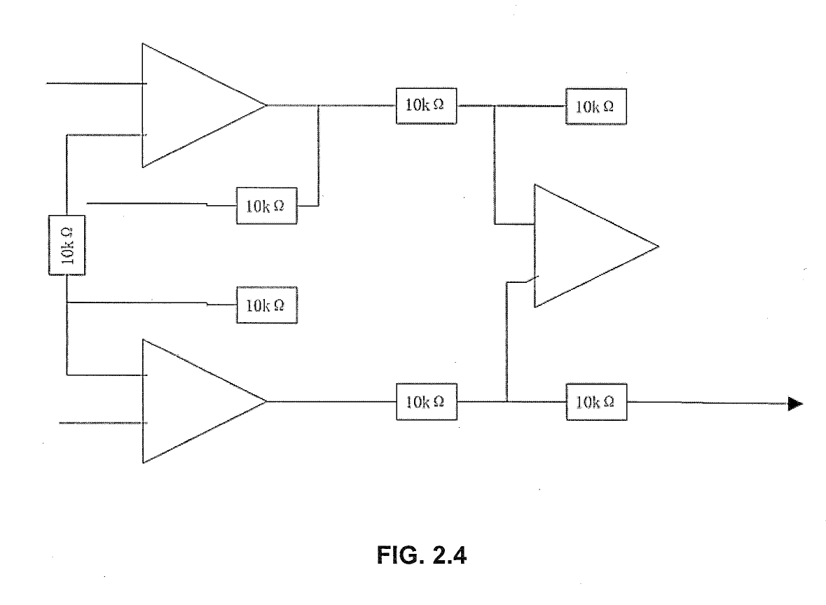

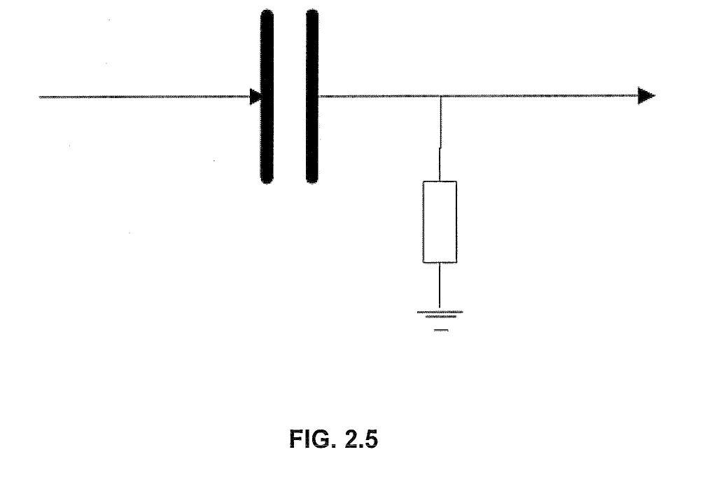

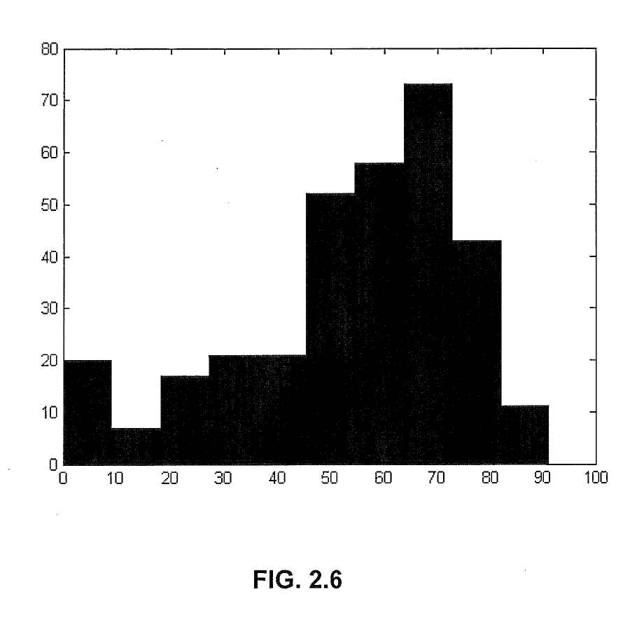

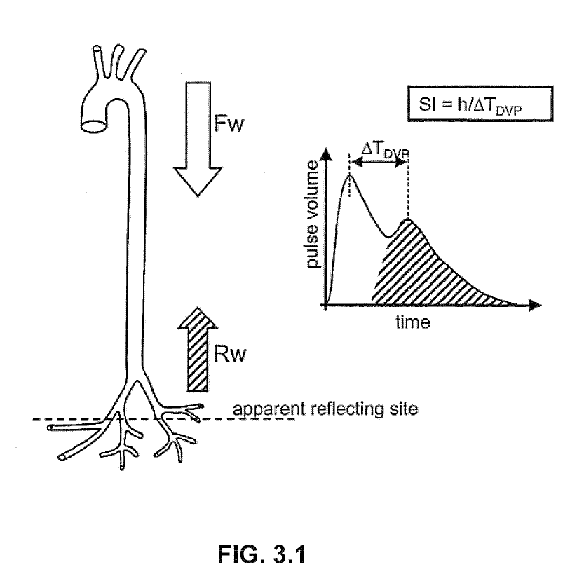

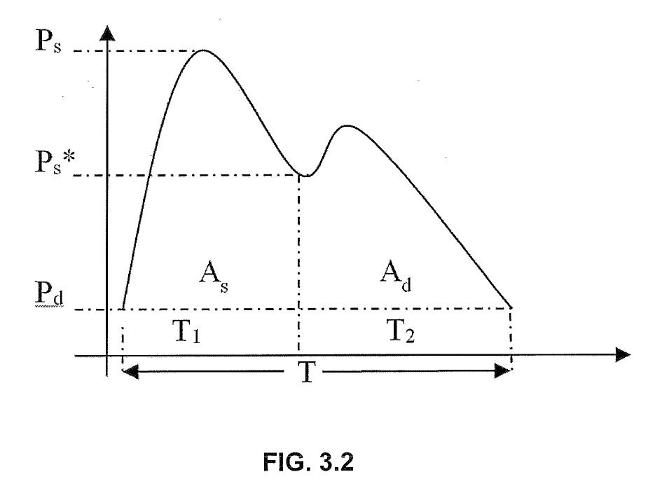

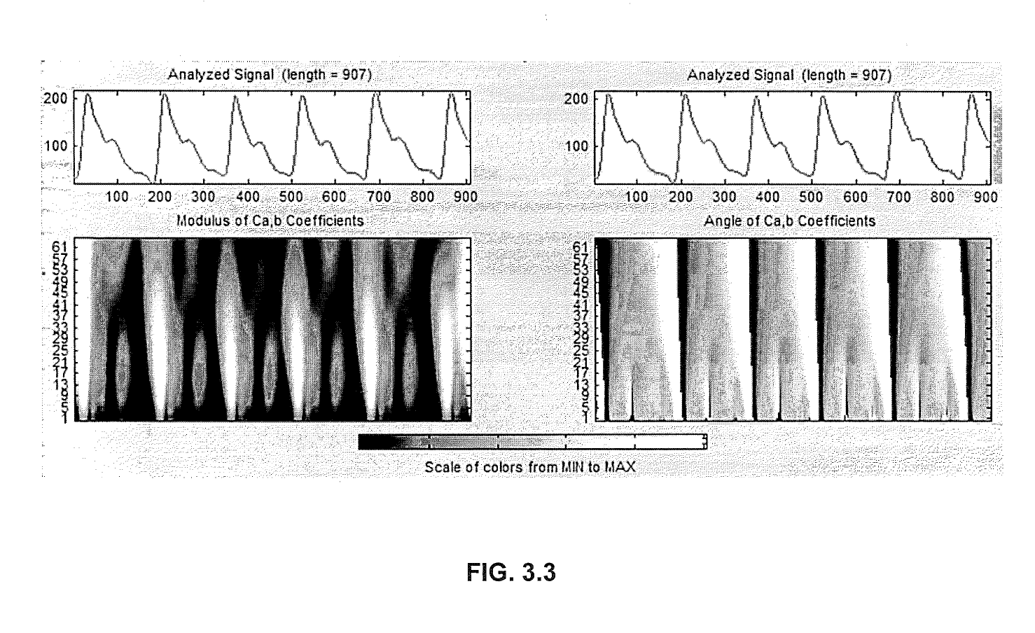

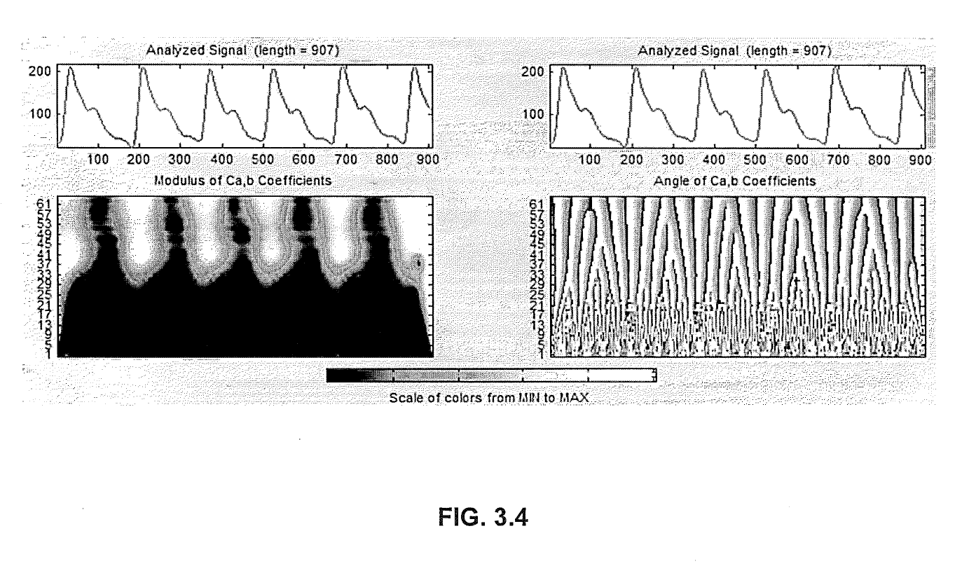

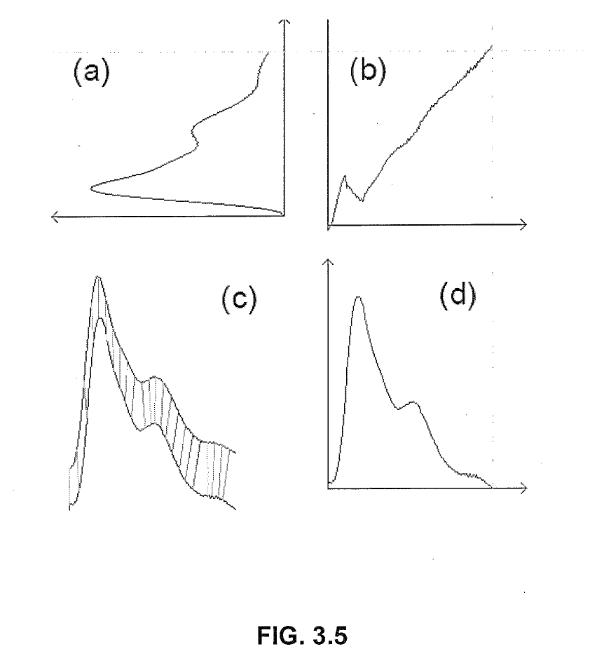

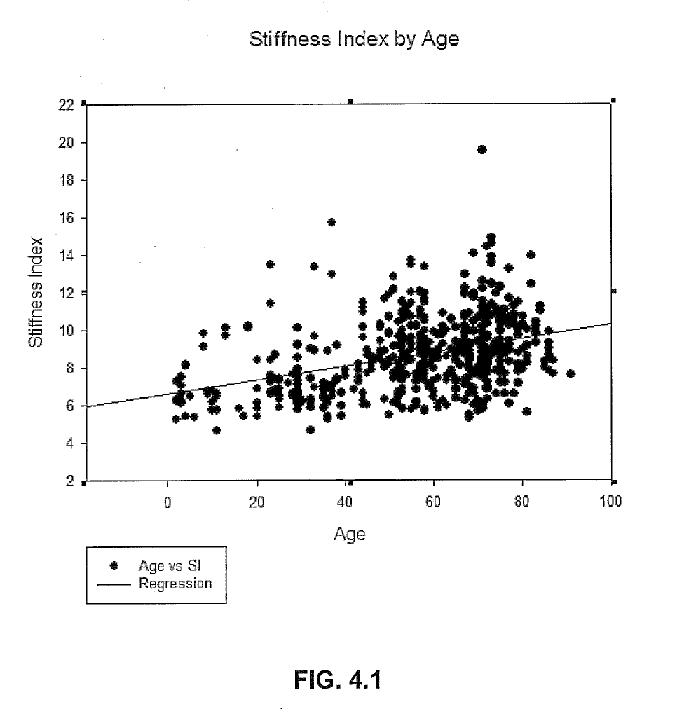

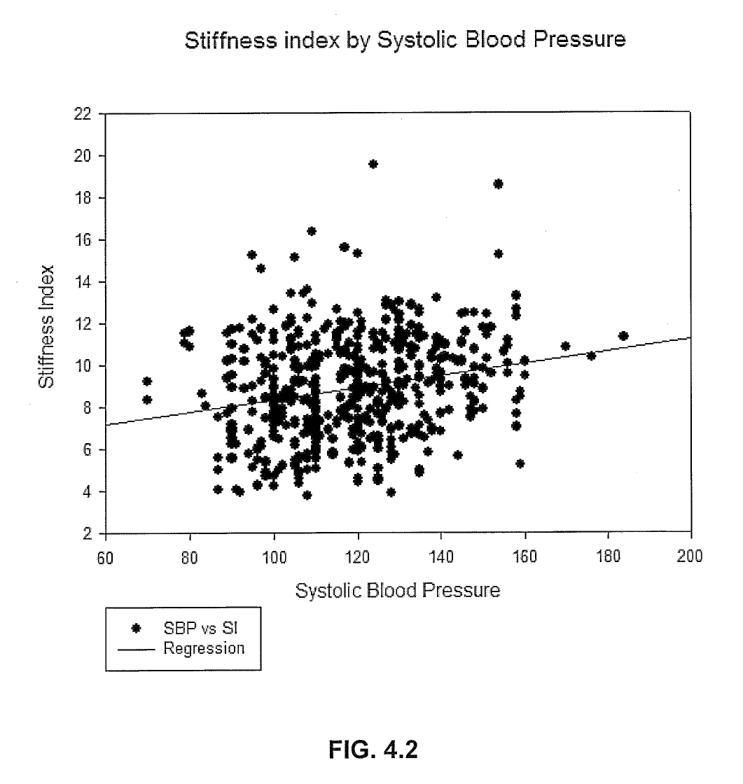

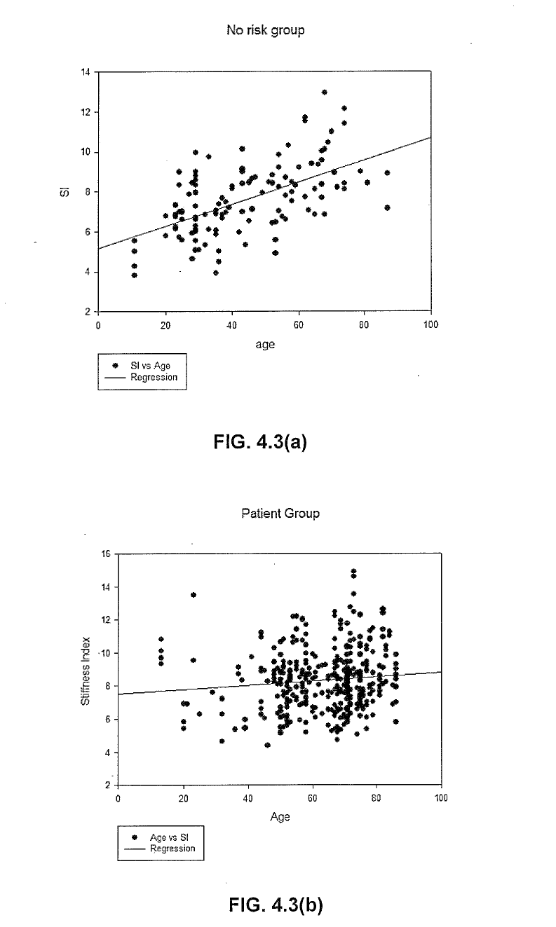

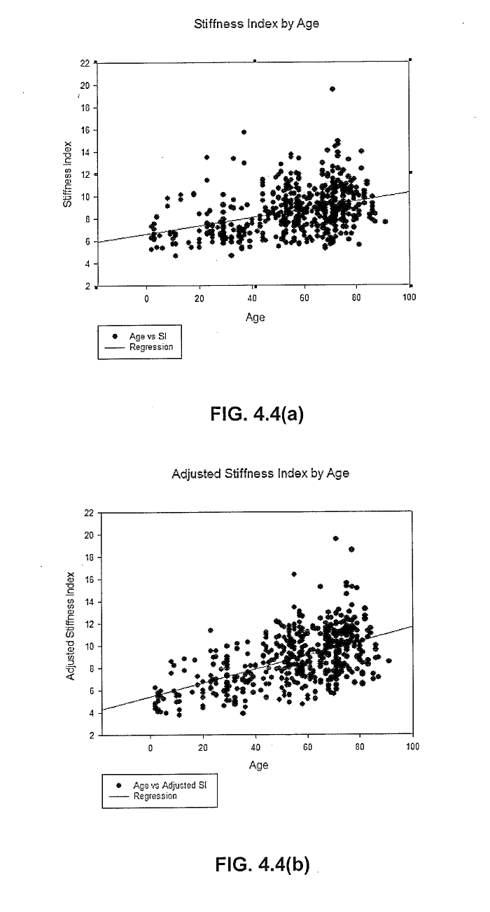

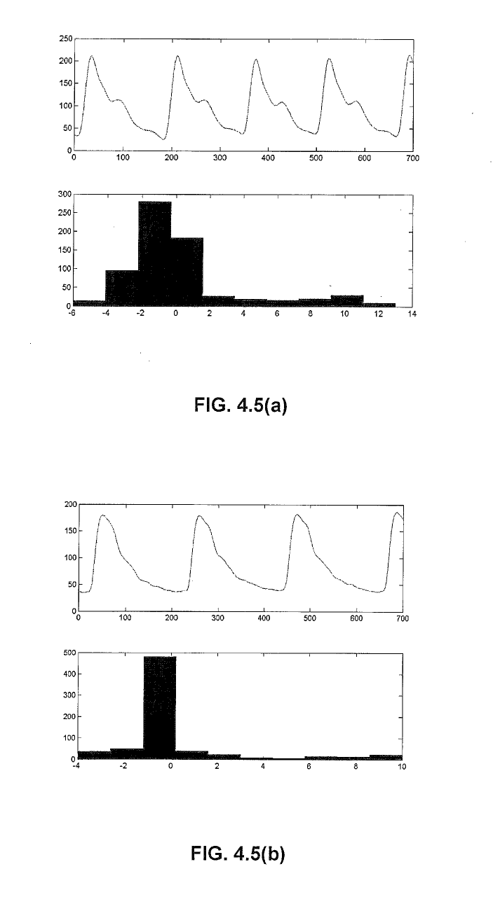

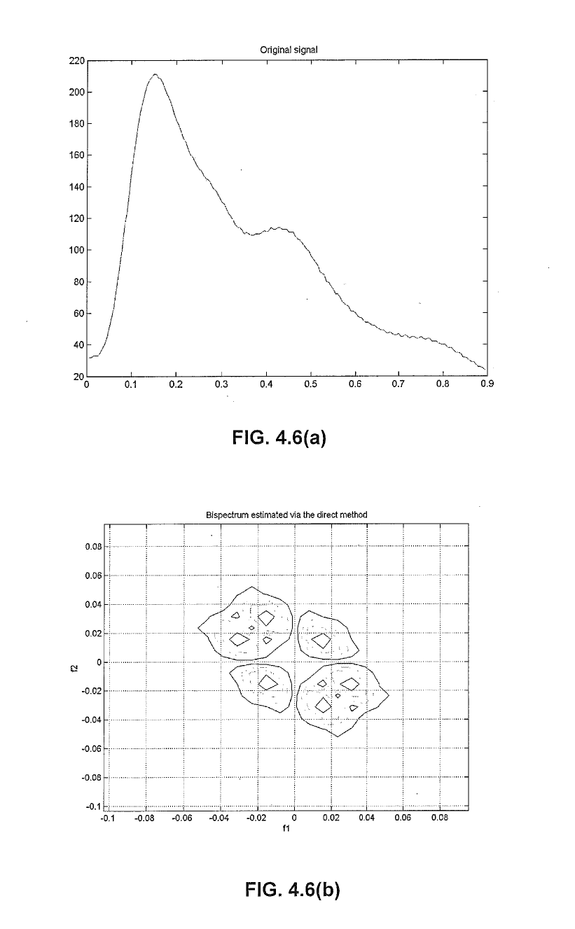

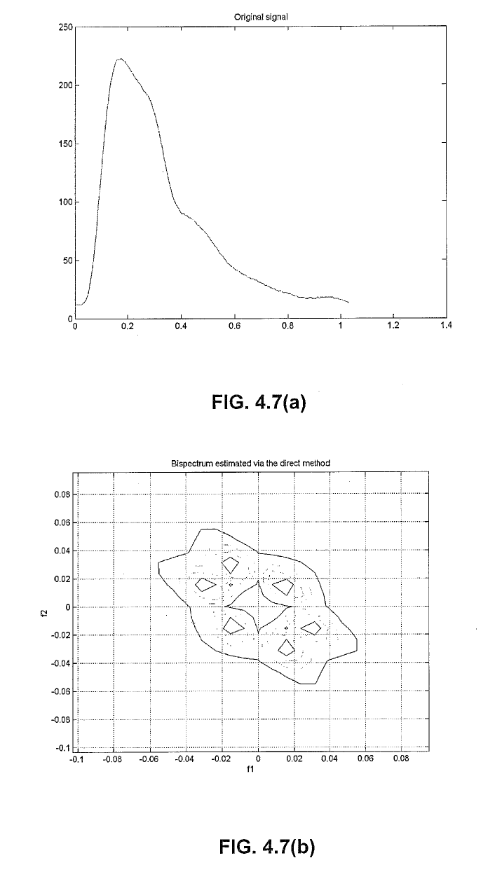

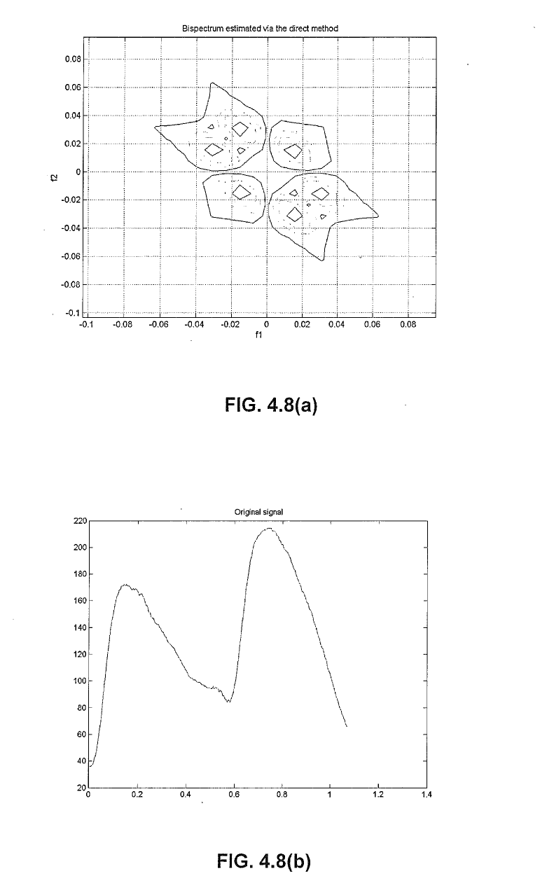

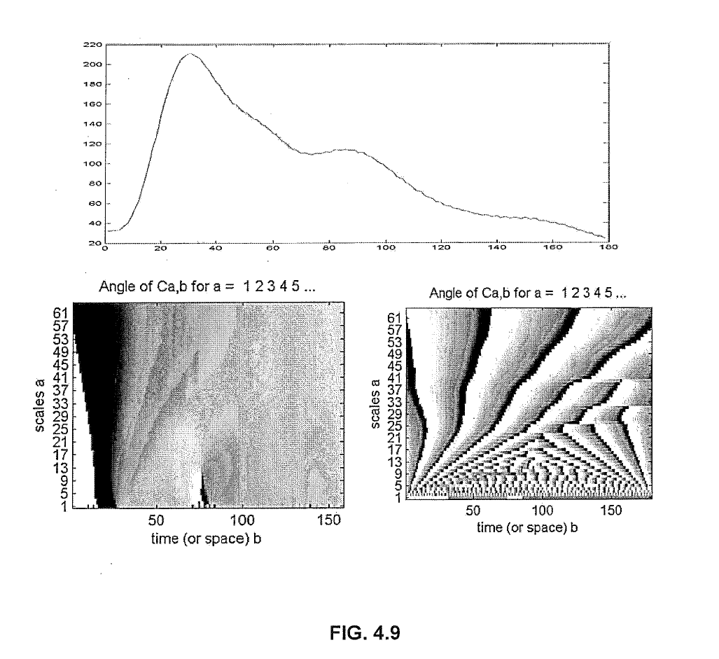

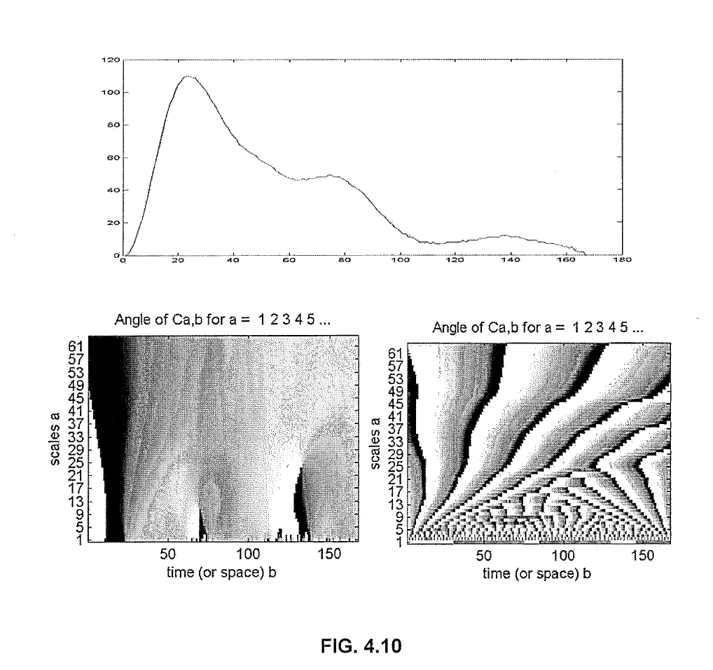

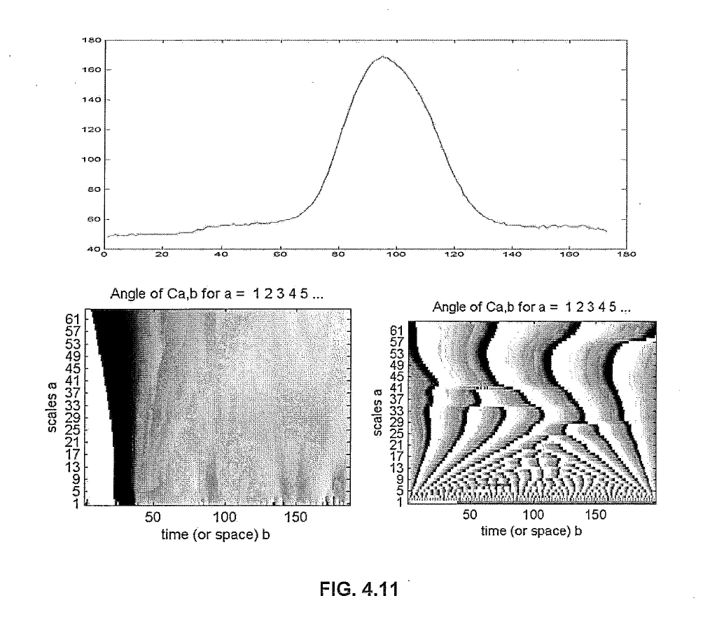

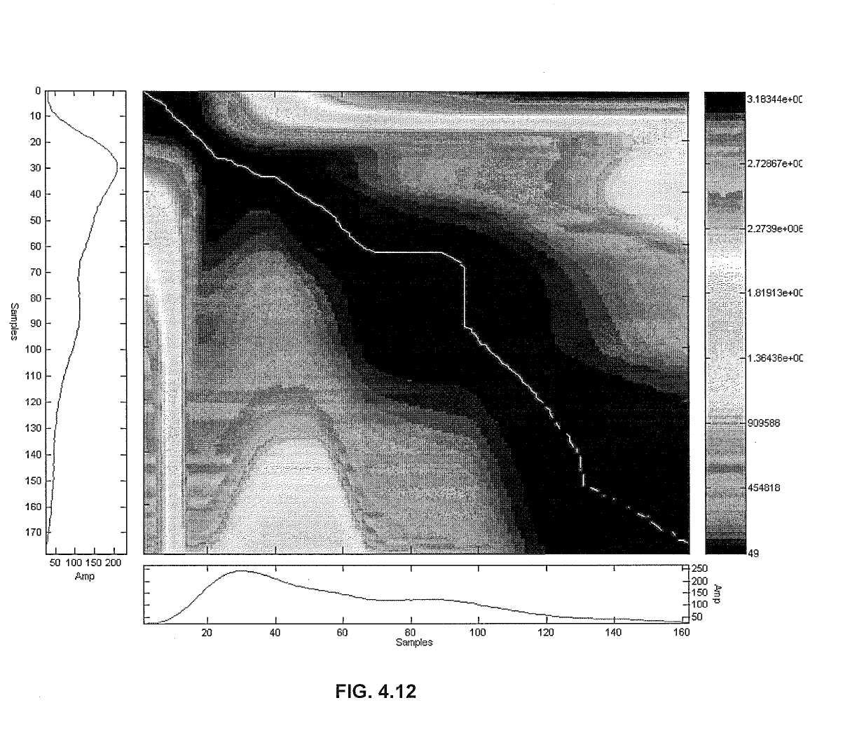

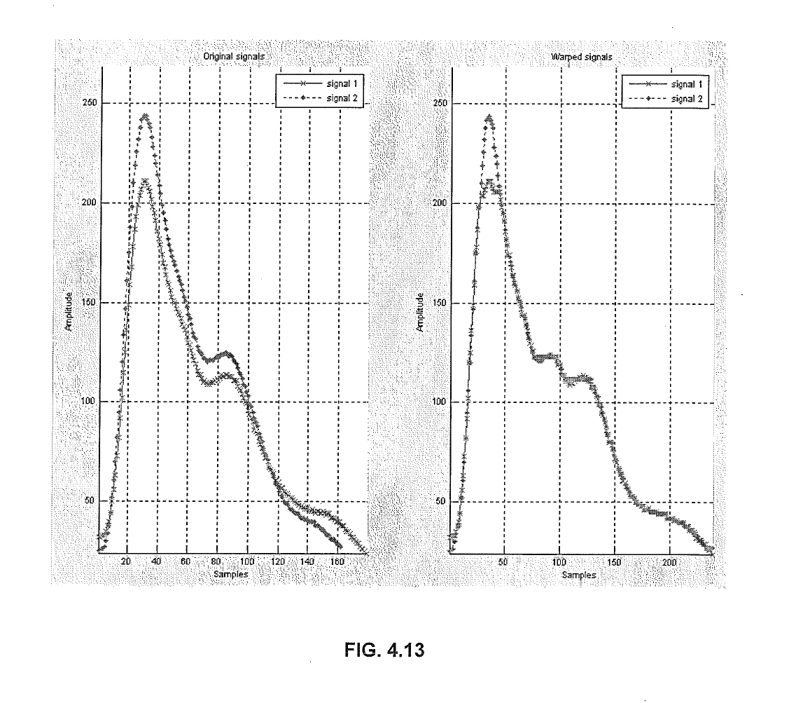

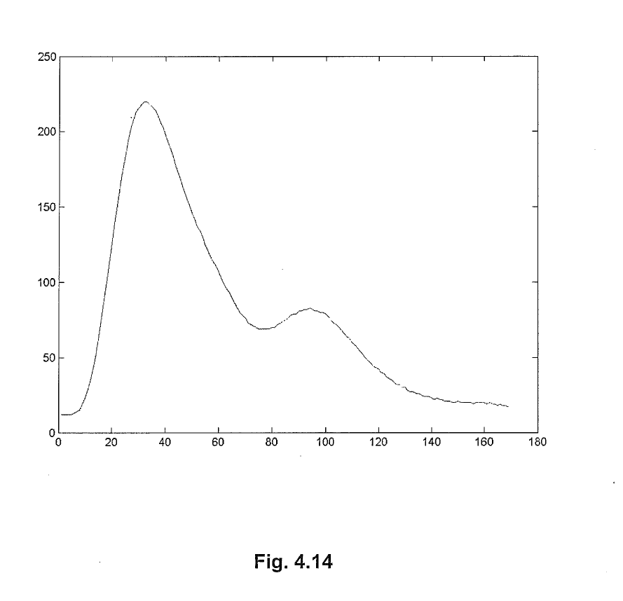

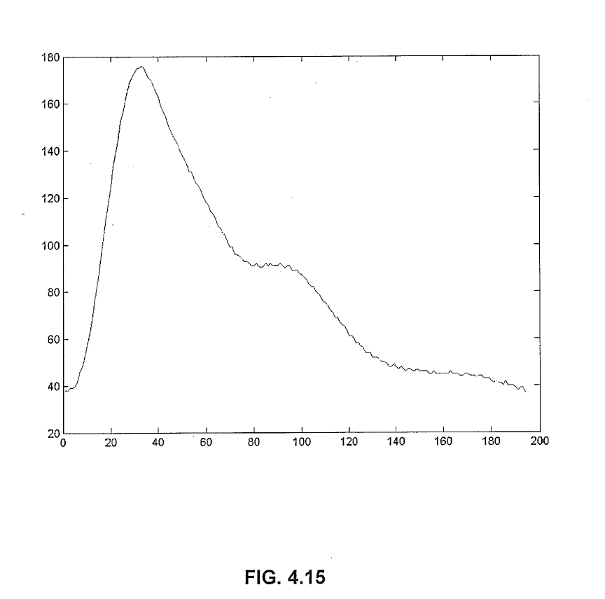

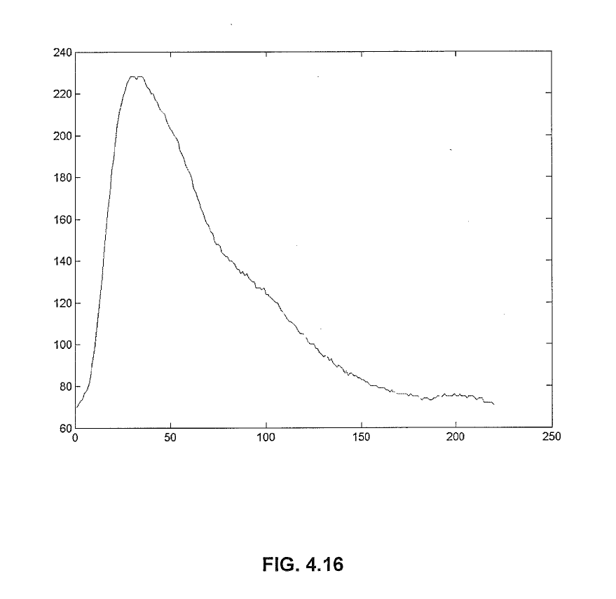

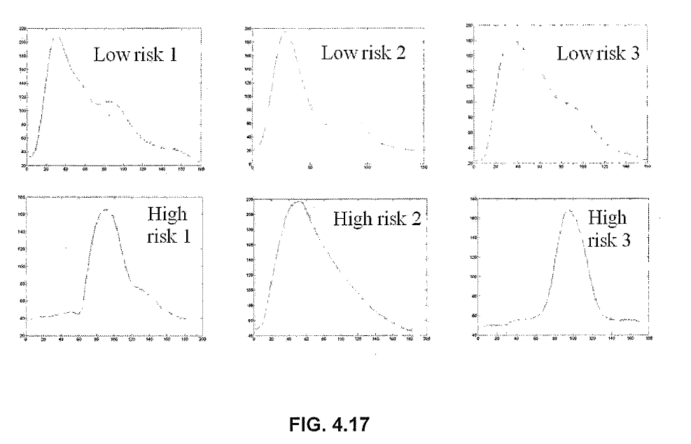

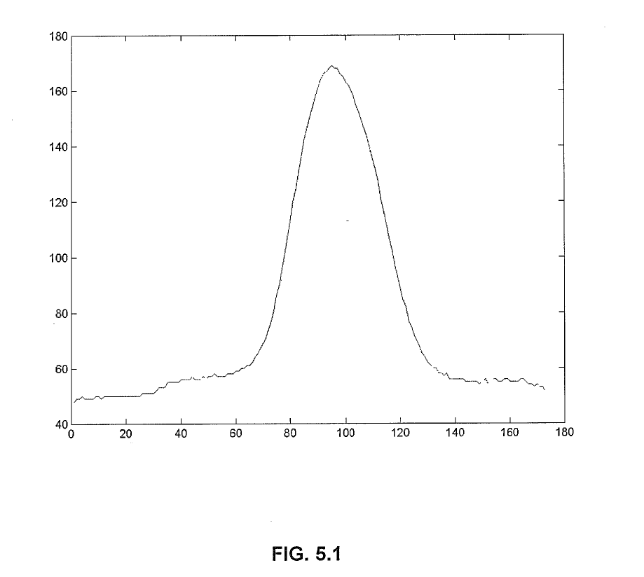

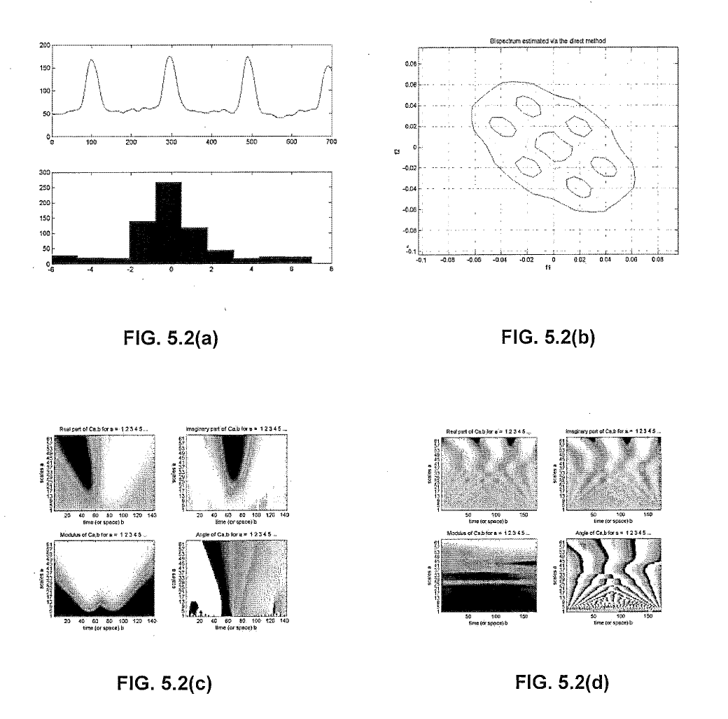

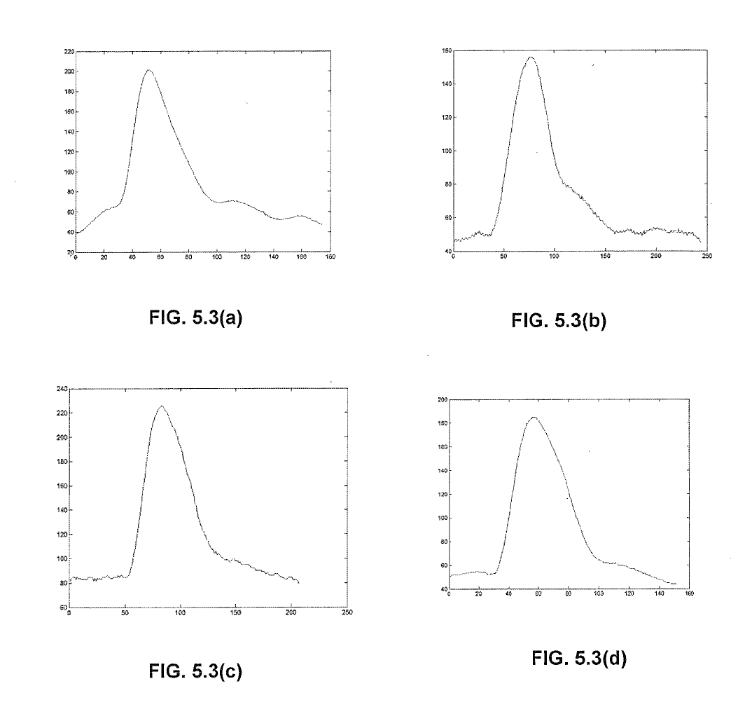

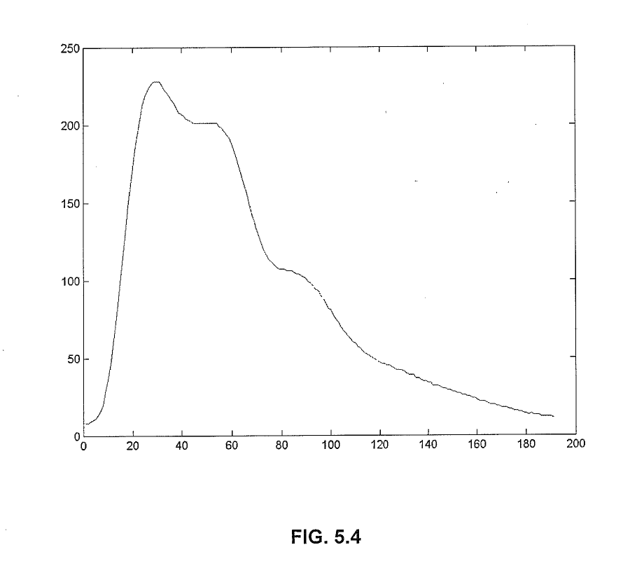

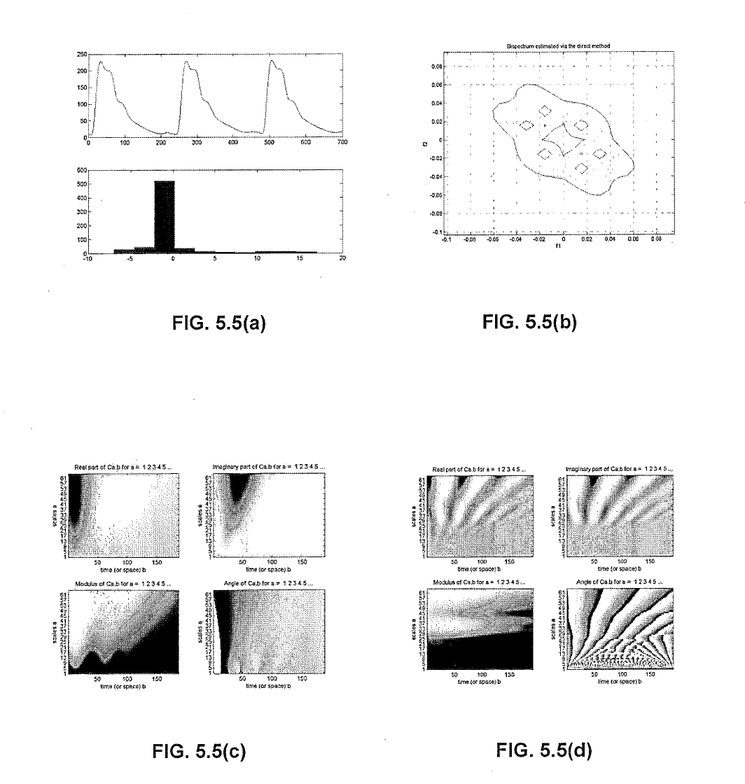

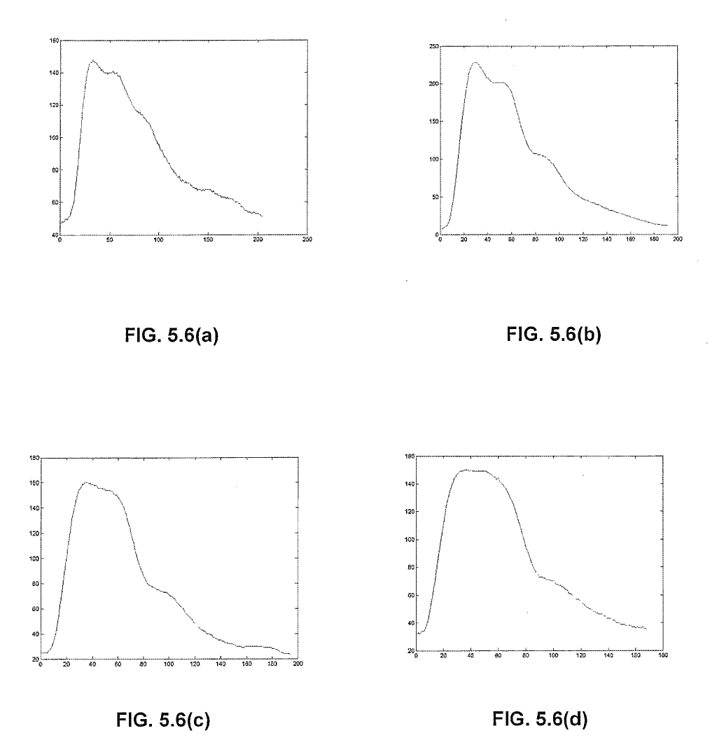

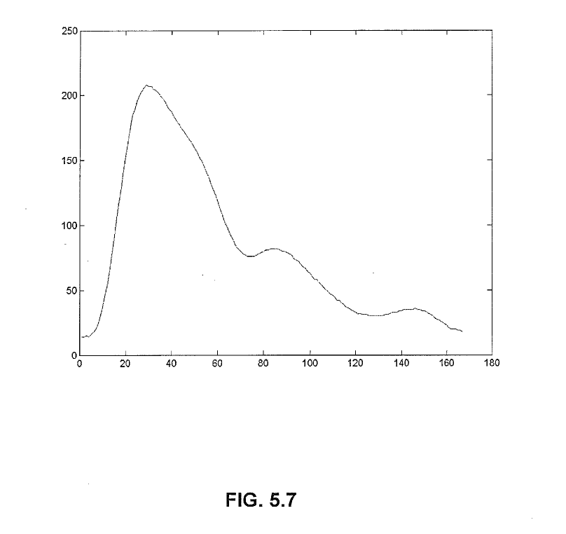

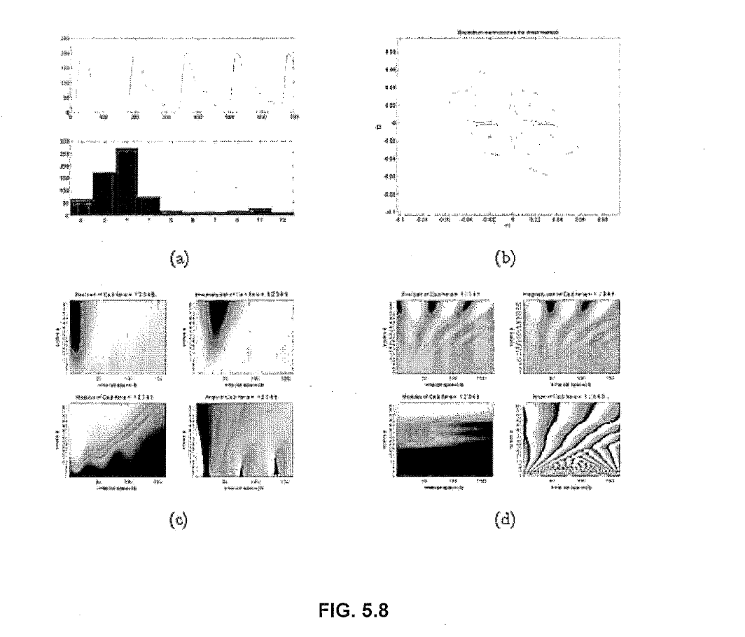

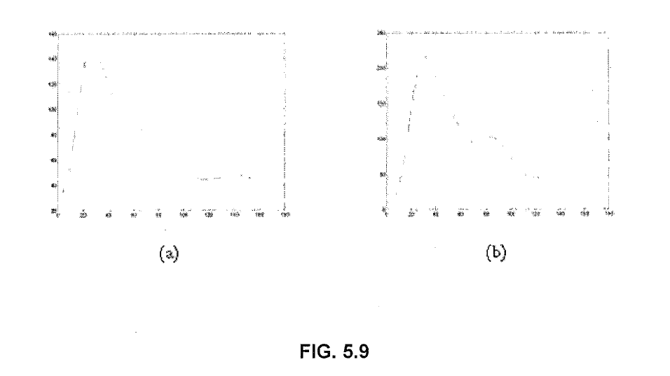

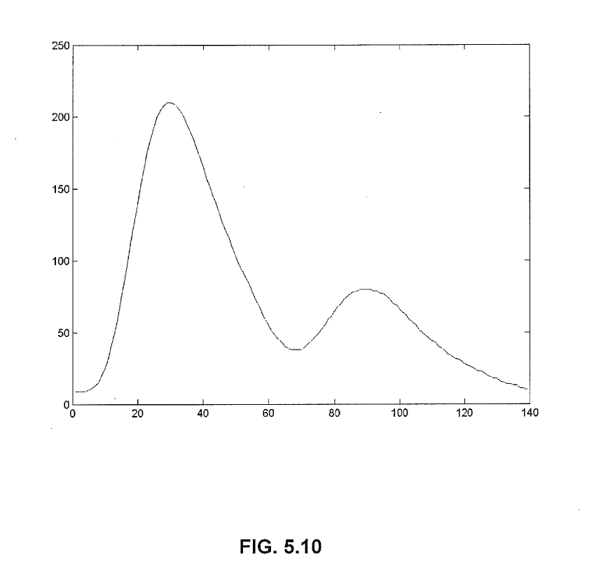

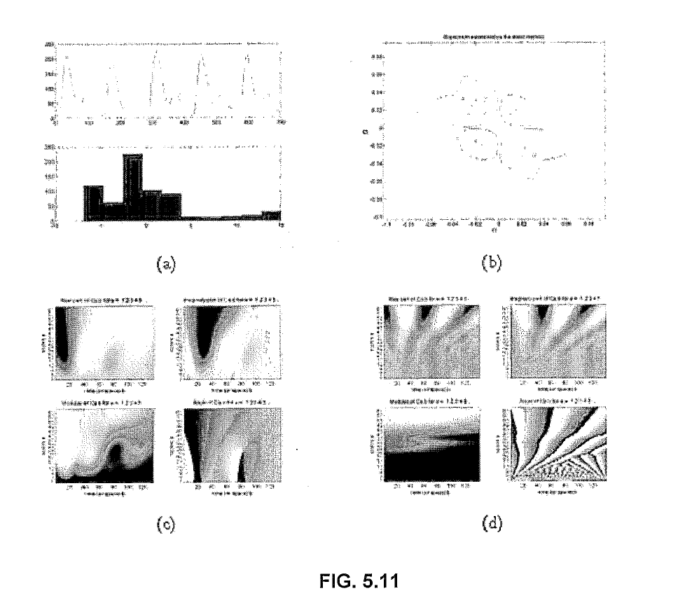

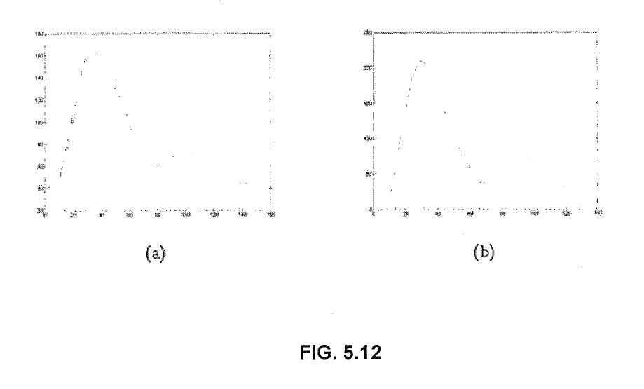

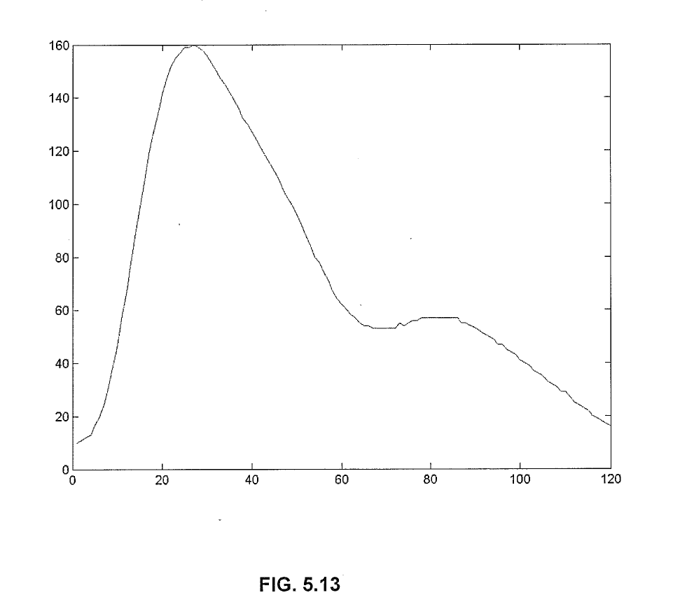

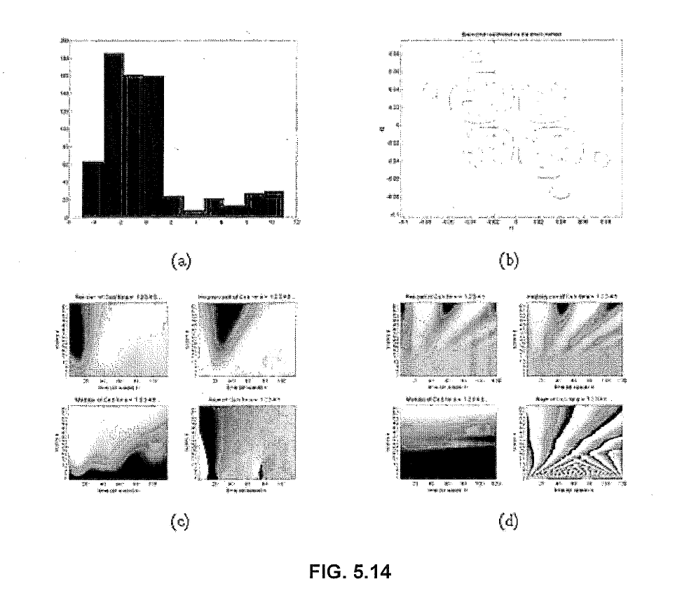

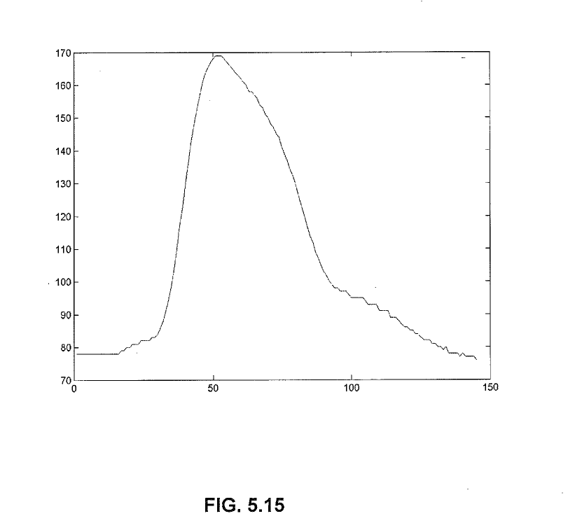

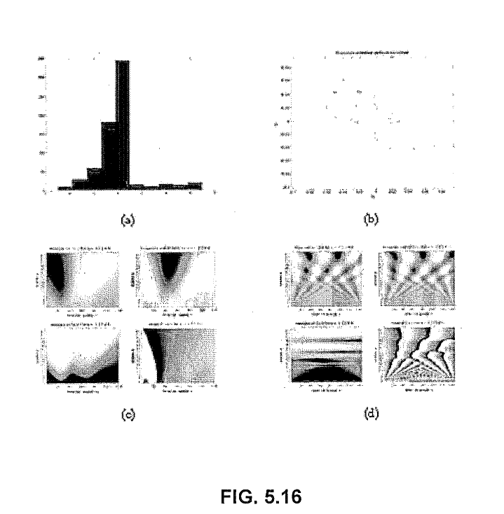

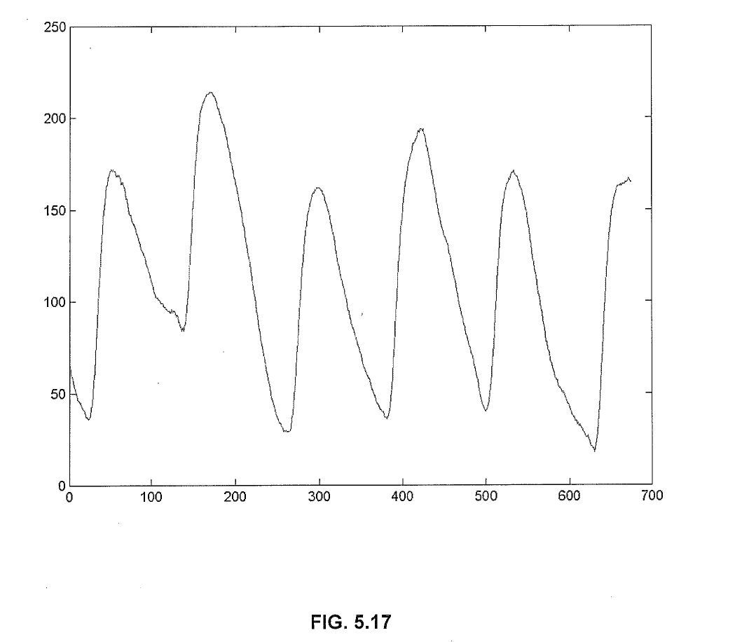

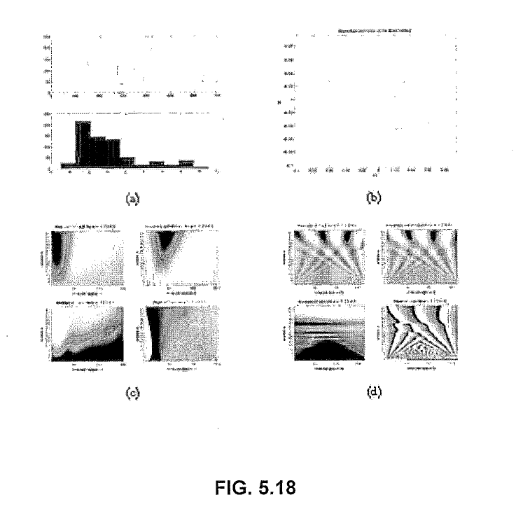

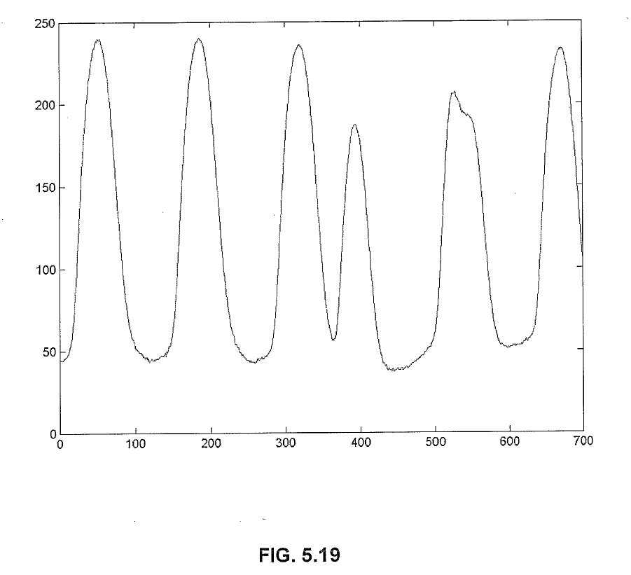

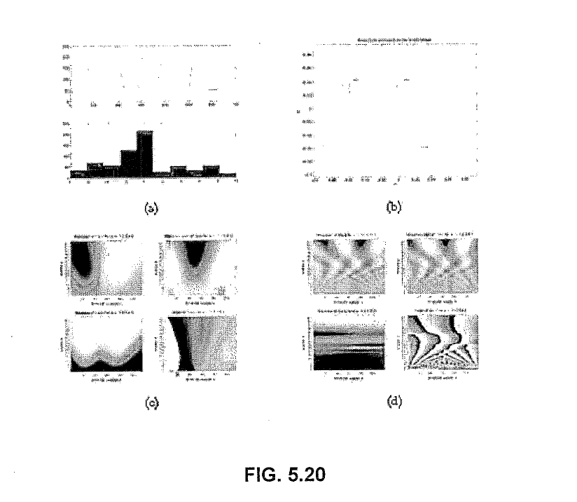

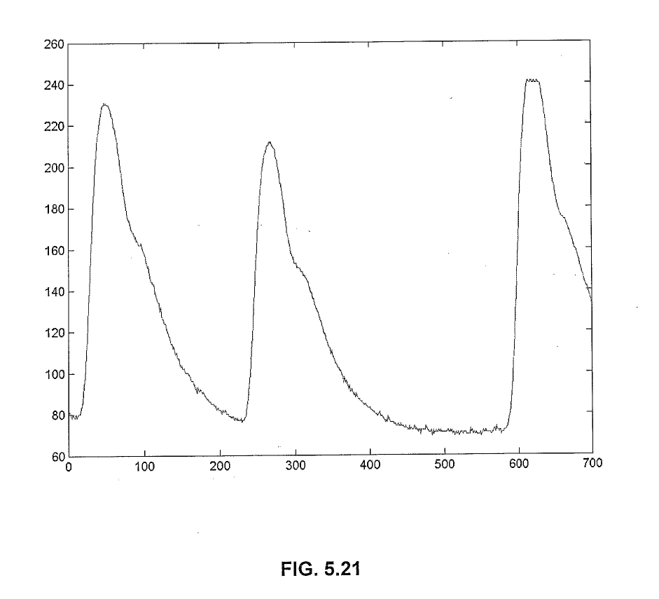

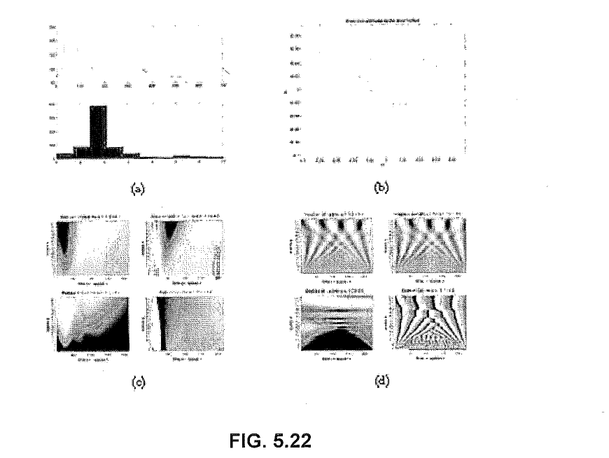

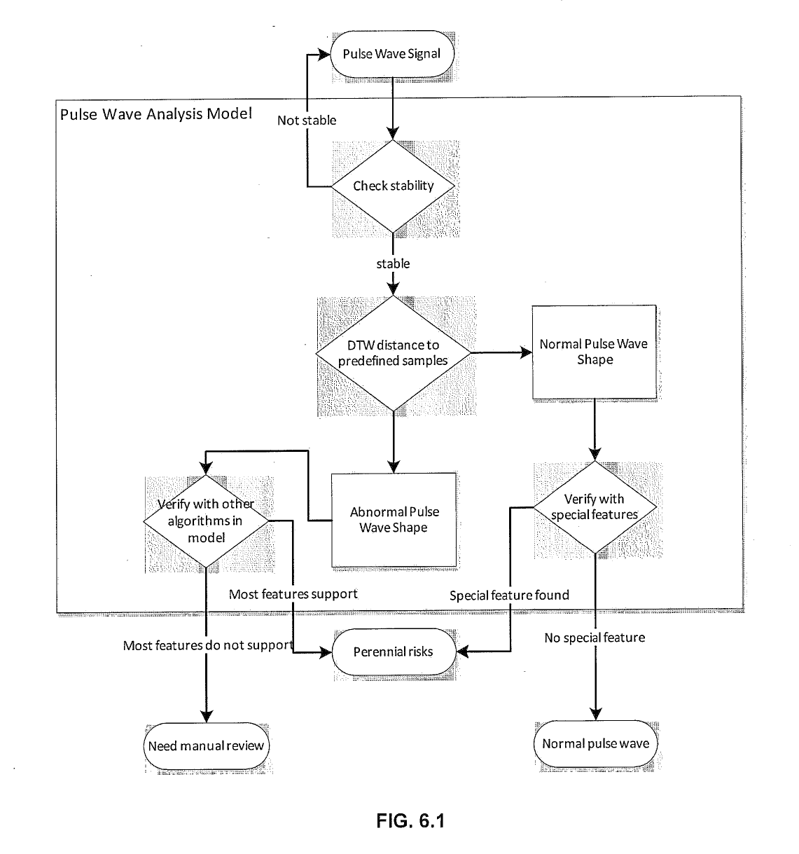

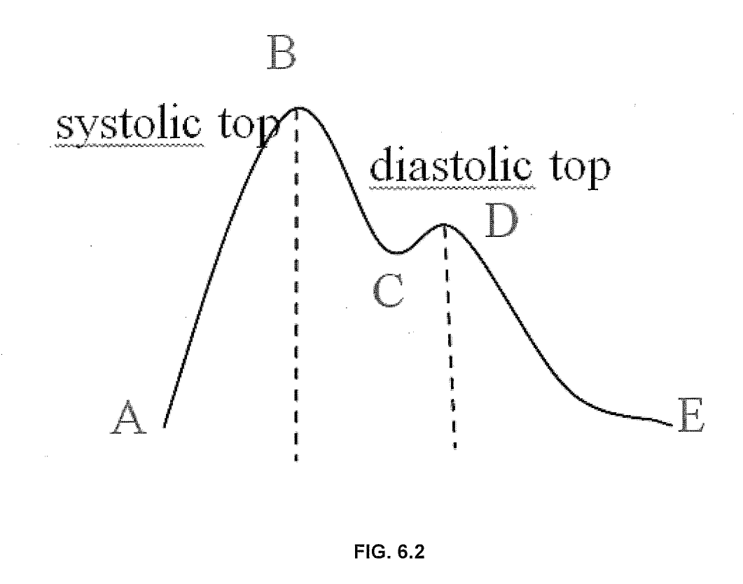

As shown in Section 3.3.4 the weighting coefficient w(k) may be an array of values with the same size as the sample pulse wave data. The vector may begin with a basic value of 1, having different weighting coefficients based on research topics, waveform components, and other conditions such as the type of simple waveform. Referring to FIG. 6.2, values may be assigned, for example, as follows:
Cardiac function: every point from A to B may have high weighting coefficient eg. 5.
Arterial Stiffness: every point from C to D may have higher weighting coefficient eg. 10
Health condition of arteries: every point from B to E may have high value eg. 2

Every point in the weighting coefficient array may have a different value though it may not be necessary to do so.

Theoretically, this method will amplify the differences of a specific part of the pulse waveform. This difference can help identify the degree of similarity between a sample waveform and a testing waveform.

Testing was done when studying arterial stiffness cases. Ten similar waveforms were used as samples in the test, three of them having a medical history of cardiac risks. DTW (Dynamic Time Warping) and Weighted DTW were performed between each sample waveform and a normal testing waveform. A Weighting coefficient of ten was applied on the diastolic components. All Weighted DTW have a higher value than DTW. The Weighted DTW result of the three high risk waveform increased more significantly than any other normal waveform, confirming the utility of the method.

| | | Difference Between | |
|---|---|---|---|
| Normal testing | | DTW and Weighted | |
| waveform | Weighted DTW | DTW | Result |
| Normal sample | Higher value than | lower difference | result in higher |
| | DTW | | similarity |
| risk sample | Higher value than | higher difference | result in lower |
| | DTW | | similarity |

Thus, the Weighted DTW provides superior analysis results than DTW.

Many variations to the described system are possible. Examples of variations include:
recording and analyzing the evolution of pulse wave data over time;
identifying evidence-tracking phenomenon;
complementing the reference value database with outcome data, which is available from many component studies; and
using reference values as cut-off values for treatment.

Other changes and variations also follow logically from the description herein. For example, the methods and systems described herein could be combined with other cardiovascular testing and analysis methods, systems and devices.

### CONCLUSIONS

One or more currently preferred embodiments have been described by way of example. It will be apparent to persons skilled in the art that a number of variations and modifications can be made without departing from the scope of the invention as defined in the claims.

All citations are hereby incorporated by reference.

What is claimed is:

1. A method of detecting cardiovascular disease comprising:
collecting and storing cardiovascular pulse wave data over time; and
performing factor-based analysis and/or waveform-based analysis of said stored cardiovascular pulse wave data.

2. The method of claim 1 wherein the factor-based analysis comprises executing a stiffness index algorithm.

3. The method of claim 1 wherein the factor-based analysis comprises executing a stiffness index algorithm adjusted for pulse rate.

4. The method of claim 3 wherein the adjusted stiffness is equal to (stiffness index multiplied by 60)/pulse rate.

5. The method of claim 1 wherein the factor-based analysis comprises executing a cardiac output algorithm.

6. The method of claim 1 wherein the waveform-based analysis comprises executing a histogram algorithm.

7. The method of claim 1 wherein tie waveform-based analysis comprises executing a bispectrum estimation algorithm.

8. The method of claim 1 wherein the waveform-based analysis comprises executing a wavelet algorithm.

9. The method of claim 1 wherein the waveform-based analysis comprises executing a Morlet wavelet algorithm.

10. The method of claim 1 wherein the waveform-based analysis comprises executing a weighted dynamic time warping a algorithm.

11. The method of claim 1 wherein the waveform-based analysis comprises:
assigning a higher weight vector to the diastolic component if the sample waveform belongs to the coronary artery disease category;
assigning a higher weight vector to the systolic component if the sample waveform belongs to the heart failure category; and
then executing a dynamic time warping algorithm.

12. The method of claim 1 wherein the waveform-based analysis comprises executing a nonlinear pattern recognition algorithm.

13. The method of claim 1 wherein the waveform-based analysis comprises performing a similarity analysis to compare stored cardiovascular pulse wave data to well-classified sample waveforms.

14. The method of claim 1 wherein the waveform-based analysis comprises evaluating the shape of the systolic component and the shape of the diastolic component separately.

15. A system for performing cardiovascular analysis comprising:
a pulse-sensing device;
an analogue to digital convertor for converting the infra-red signal to a digital signal;
a USB interface for communicating said digital signal to a computing device;
said computing device being operable:
to receive and store digitized cardiovascular pulse wave data over time, from said USB interface; and
to perform factor-based analysis and/or waveform-based analysis of said digitized cardiovascular pulse wave data.

16. The system of claim 15, further comprising a high pass filter.

17. The system of claim 15, further comprising a wireless communication device for collecting said digital signal and transmitting it to said computing device.

18. The system of claim 15, wherein said wireless communication device comprises a Smartphone.

19. The system of claim 15, wherein said system is portable.

20. The system of claim 15, wherein said pulse-sensing device comprises a finger clip with a USB-powered infra-red transmitter and sensor pair.

21. The system of claim 15, wherein said a pulse-sensing device comprises a wrist pressure sensor.

22. The system of claim 15, wherein said computing device is operable to reject unstable data.

23. A system for detecting cardiovascular disease comprising:
a pulse-sensing device;
a computing device being operable:
to receive cardiovascular pulse wave data from said pulse-sensing device; and
to perform factor-based analysis of said cardiovascular pulse wave data.

24. The method of claim 10 wherein the weighted dynamic time warping algorithm comprises performing a wave similarity analysis where portions of a waveform are assigned different weights.
